(19) United States
(12) Patent Application Publication
(10) Pub. No.: US 2021/0145955 A1
(43) Pub. Date: May 20, 2021

(54) CLOSTRIDIAL NEUROTOXIN FORMULATIONS AND USE

(71) Applicant: Bonti, Inc., Madison, NJ (US)

(72) Inventors: Kenton Abel, Madison, NJ (US); Michael Jarpe, Madison, NJ (US); Fauad Hasan, Madison, NJ (US)

(21) Appl. No.: 16/624,530

(22) PCT Filed: Jun. 26, 2018
(86) PCT No.: PCT/US2018/039466

§ 371 Date: Dec. 19, 2019

Related U.S. Application Data
(60) Provisional application No. 62/548,501, filed on Aug. 22, 2017.
(60) Provisional application No. 62/533,211, filed on Jul. 17, 2017.
(60) Provisional application No. 62/525,030, filed on Jun. 26, 2017.

(51) Int. Cl. A61K 39/08; A61K 9/00; A61K 31/451; A61P 23/02; A61P 21/02

(52) U.S. Cl. CPC A61K 39/08; A61K 9/0019; A61K 9/0053; A61K 2039/545; A61P 23/02; A61P 21/02; A61K 31/451

(57) ABSTRACT

Disclosed herein are compositions and methods for use in neurotoxin treatments.

### FIELD

The present specification relates to the use of neurotoxin formulations.

### BACKGROUND

Botulinum toxins are the most potent protein toxins. They act by blocking acetylcholine release at the neuromuscular junction, resulting in denervation of muscles. Botulinum toxins also have activity at other peripheral cholinergic nerve terminals and lead, for example, to reduced salivation or sweating and to diminished facial lines and wrinkles.

The botulinum toxins are synthesized and released by certain Clostridium species in the form of large complexes comprising the botulinum toxin molecule (the “neurotoxic component”) and associated non-toxic bacterial proteins (also referred to as “complexing proteins”).

Regardless of serotype, the molecular mechanism of toxin intoxication appears to be somewhat similar, and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The catalytic domain (light chain) then translocates through the endosomal membrane into the cytoplasm.

Opioids are a class of drugs that includes heroin, synthetic opioids such as fentanyl, and pain relievers available by prescription, such as oxycodone, codeine, morphine, and many others. These drugs are chemically related and interact with opioid receptors on nerve cells in the body and brain. Opioid pain relievers are generally safe when taken for a short time and as prescribed by a doctor, but because they produce euphoria in addition to pain relief, they can be misused (taken in a different way or in a larger quantity than prescribed, or taken without a doctor's prescription). Regular use—even as prescribed by a doctor—can lead to dependence and, when misused, opioid pain relievers can lead to overdose incidents and deaths.

Anesthesia is commonly employed to control pain, for example in connection with a surgical procedure. Local anesthesia numbs a small part of the body. Regional anesthesia blocks pain to a larger part of your body. General anesthesia affects the brain and the rest of your body.

### SUMMARY

Disclosed herein are compositions and methods comprising neurotoxins and the use thereof in combination with, or as a replacement for, opioids, for example reduce pain. Disclosed embodiments comprise use of a “fast-acting” botulinum toxin in combination with an opioid. Disclosed embodiments comprise use of a “fast-acting” botulinum toxin instead of an opioid, for example in conjunction with an opioid withdrawal treatment.

Disclosed herein are compositions and methods comprising neurotoxins and the use thereof in combination with anesthetics to reduce pain both during surgery as well as in the post-surgical period. For example, disclosed embodiments comprise use of a “fast-acting” botulinum toxin in combination with an anesthetic.

Disclosed herein are compositions and devices for administration of neurotoxins. Compositions disclosed herein can comprise multiple types of neurotoxins, for example multiple Clostridial neurotoxins. This provides compositions that combine advantageous features from different neurotoxins. Devices disclosed herein can administer multiple types of neurotoxins simultaneously. Disclosed formulations comprise a combination of botulinum neurotoxin serotypes, for example types A and E, types A and B, Types E and B, and the like.

Disclosed herein are compositions and methods for use in initiating neurotoxin treatment, for example clostridial toxin, for example botulinum toxin, for example botulinum toxin type E, in a patient. For example, disclosed embodiments comprise use of a “fast-acting” botulinum toxin.

In embodiments, the botulinum toxin is a “fast-recovery” toxin.

In embodiments, the “fast-acting” botulinum toxin is also a “fast-recovery” toxin.

Disclosed embodiments comprise wild-type neurotoxins, for example wild-type clostridial neurotoxins, for example wild-type botulinum type E.

In embodiments, the patient is neurotoxin naïve.

In embodiments, the patient is clostridial toxin naïve.

In embodiments, the patient is botulinum toxin naïve.

In embodiments, the patient is botulinum type E (BoNT/E) naïve.

In embodiments, the patient is botulinum type A (BoNT/A) naïve.

In embodiments, the patient is botulinum type B (BoNT/B) naïve.

In embodiments, the patient is “fast-acting” neurotoxin naïve.

In embodiments, the patient is “fast-recovery” neurotoxin naïve.

Disclosed herein are compositions and methods for use in minimizing scarring. For example, disclosed embodiments comprise use of a fast-acting botulinum toxin to reduce muscle tension in the proximity of a wound, thus preventing or reducing scarring. In embodiments, muscle activity in the proximity of a skin incision or laceration is reduced, thus reducing or preventing scar formation.

Disclosed herein are compositions and methods for use in cosmetic treatments. For example, disclosed embodiments comprise use of a fast-acting botulinum toxin to treat, for example, glabellar lines.

In embodiments, disclosed methods comprise additional surgical procedures, for example cosmetic procedures. For example, disclosed embodiments comprise administration of a fast-acting botulinum neurotoxin in combination with, for example, a dermal filler injection, an eye lift, rhinoplasty, or the like.

In embodiments, the cosmetic treatment can comprise a supplemental botulinum administration after an initial administration.

In embodiments, disclosed methods comprise administration of a fast-acting botulinum neurotoxin in combination with, for example, a slower-acting neurotoxin.

In embodiments, disclosed methods comprise administration of a fast-recovery botulinum neurotoxin in combination with, for example, a slower-recovery neurotoxin.

In embodiments, disclosed methods comprise administration of a fast-acting botulinum neurotoxin in combination with, for example, a slower-recovery neurotoxin.

In embodiments, neurotoxin dosage, for example fast-acting botulinum toxin such as BoNT/E dosage, is expressed in protein amount.

### BRIEF DESCRIPTION OF THE DRAWINGS

FIG. 1 depicts injection sites used in a cosmetic surgery procedure.

FIG. 2 shows primary efficacy of a glabellar line treatment study.

FIG. 3 shows secondary efficacy of a glabellar line treatment study.

FIG. 4 shows the effect of a single local administration of a disclosed type E botulinum composition in a rat model of post-operative pain.

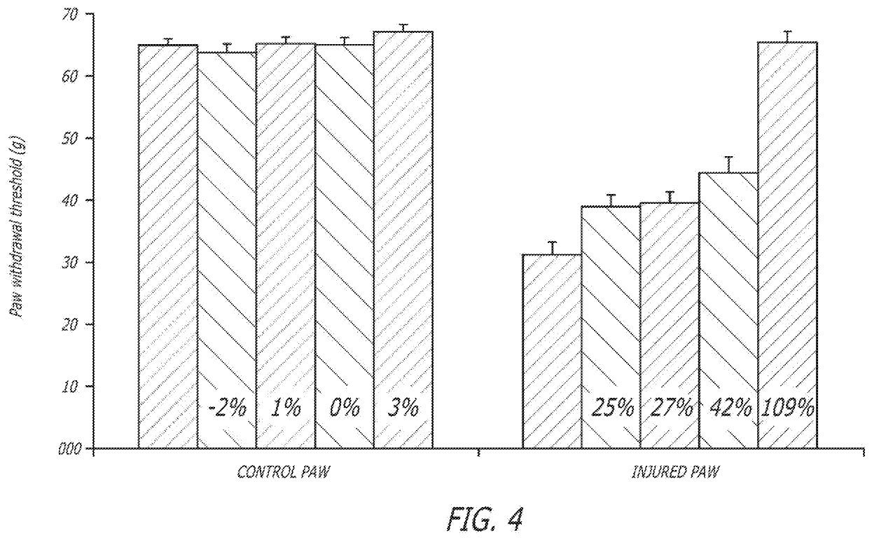

### DETAILED DESCRIPTION

Embodiments comprise use of neurotoxins, for example in single- or multiple-type neurotoxin compositions, in combination with, for example, other active agents such as opioids or anesthetics.

Embodiments disclosed herein can reduce local muscle activity, nerve activity, and pain sensation. This reduction can aid in treatment and recovery, for example recovery following a surgical procedure. In embodiments the surgical procedure can comprise any intentional disruption to the body, for example cosmetic surgery, dental surgery, and the like.

Embodiments disclosed herein can reduce local muscular activity and thereby reduce the appearance of cosmetic imperfections or irregularities, for example facial lines. In embodiments the cosmetic irregularities can comprise glabellar lines, forehead lines, “bunny” lines, smile irregularities, chin irregularities, platysmal bands, “marionette” lines, lip lines, crow's feet, eyebrow irregularities, combinations thereof, and the like. Embodiments comprise methods comprising dermatological surgical procedures, such as treatment for Actinic Keratosis, Seborrheic Keratosis, Basocelular Carcinoma, Squamous Cell Carcinoma, and other lesions or subdermal cysts.

Administration sites useful for practicing disclosed embodiments can comprise any area where muscle and/or nerve activity is to be reduced. For example, in the case of facial pain, for example due to facial surgery, disclosed embodiments can comprise administration to the glabellar complex, including the corrugator supercilli and the procerus; the obicularis oculi; the superolateral fibers of the obicularis oculi; the frontalis; the nasalis; the levator labii superioris aleque nasi; the obicularis oris; the masseter; the depressor anguli oris; and the platysma.

In the case of pain in the trunk region, for example due to a surgical procedure, disclosed embodiments can comprise administration to, for example, the external intercostals, the internal intercostals, the transverse abdominis, the Infraspinatus, the rectus abdominis, the serratus anterior, the diaphragm, or combinations thereof.

In the case of pain in the upper extremities, for example due to a surgical procedure, disclosed embodiments can comprise administration to, for example, the pectoralis major, the latissimus dorsi, the deltoid, the teres major, the biceps brachii, the triceps brachii, the brachialis, the brachioradialis, the palmaris longus, the flexor carpi radialis, the flexor digitorum superficialis, the extensor carpi radialis, the extensor digitorum, the extensor digiti minimi, the extensor carpi, the ulnaris, or combinations thereof.

In the case of pain in the lower extremities, for example due to a surgical procedure, disclosed embodiments can comprise, for example, administration to, for example, the iliopsoas, the sartorius, the gluteus maximus, the gluteus medius, the tensor fasciae latae, the adductor longus, the gracilis, the semimembranosus, the semitendinosus, the biceps femoris, the rectus femoris, the vastus lateralis, the vastus intermedium, the vastus medialis, the tibialis anterior, the gastrocnemius, the soleus, the peroneus longus, the peroneus brevis, or combinations thereof.

Disclosed herein are compositions and devices for administration of neurotoxins. Compositions disclosed herein can comprise multiple types of neurotoxins, for example multiple Clostridial neurotoxins. Devices disclosed herein can administer multiple types of neurotoxins simultaneously. This provides compositions that combine advantageous features from different neurotoxins, for example rapid onset of action as demonstrated by BoNT/E, as well as extended effectiveness, as demonstrated by BoNT/A. In an embodiment disclosed herein, the neurotoxin composition comprises BoNT/E and BoNT/A.

Certain patients are hesitant to begin neurotoxin treatments for various reasons, including the delay between administration and effect, as well as the possibly-long duration of effect(s). The instant disclosure provides methods and compositions for initiating neurotoxin treatments that address these concerns. For example, disclosed methods and compositions provide for a rapid effect as well as a rapid recovery. Thus, disclosed methods provide a treatment alternative to other neurotoxins, as the disclosed methods allow the patient to rapidly achieve a desired effect, while minimizing the duration of that effect. Then, if desired, the patient can elect further treatment with a neurotoxin, for example one that provides longer effect duration.

In one embodiment, administering a single composition comprising both BoNT/A and BoNT/E permits the concentration of BoNT/E to be lower than if given by itself, while still achieving the desired therapeutic effect of a more rapid onset of action than BoNT/A and a longer duration than BoNT/E. In embodiments, the neurotoxins are formulated separately and combined when administered. In embodiments, the neurotoxins are formulated together.

The neurotoxins can be made by a Clostridial bacterium, such as by a Clostridium botulinum, Clostridium butyricum, or Clostridium beratti bacterium. Additionally, the neurotoxins can be modified neurotoxins; that is a neurotoxin that has at least one of its amino acids deleted, modified or replaced, as compared to the native or wild-type neurotoxin. Furthermore, the neurotoxins can be recombinantly produced or derivatives or fragments thereof.

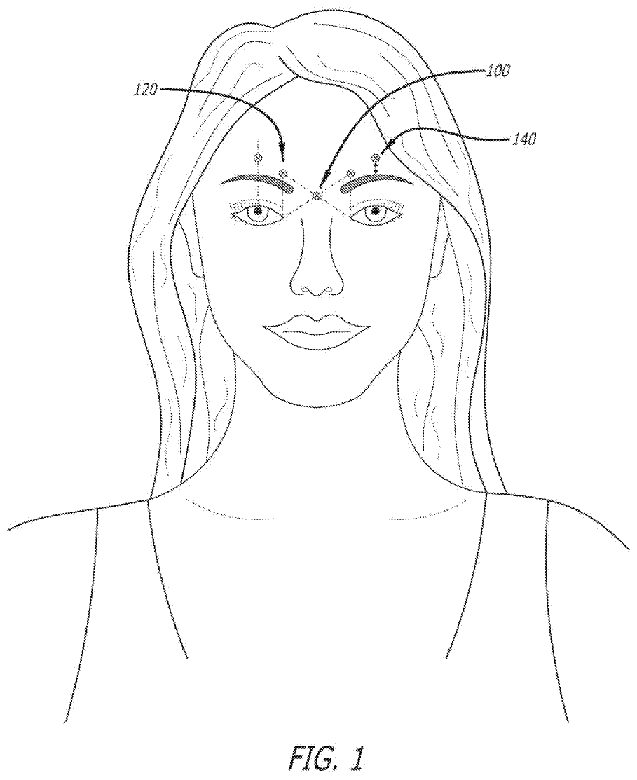

Administration sites useful for practicing the disclosed embodiments can comprise the glabellar complex, including the corrugator supercilli and the procerus; the obicularis oculi; the superolateral fibers of the obicularis oculi; the frontalis; the nasalis; the levator labii superioris aleque nasi; the obicularis oris; the masseter; the depressor anguli oris; and the platysma. Exemplary injection sites useful in glabellar line treatments are shown in FIG. 1.

Disclosed embodiments can comprise treatment of, for example, skin disorders, for example, acne, and the like. Disclosed embodiments can comprise treatment of inflammatory skin diseases. For example, disclosed embodiments can comprise treatment of psoriasis, eczema, and the like. Embodiments comprise treatment of gross wrinkles.

Embodiments disclosed herein can reduce local muscular activity and thereby reduce the development of scars, for example scars resulting from surgery. In embodiments the surgery can comprise cosmetic surgery, for example rhinoplasty, an eye lift, a “tummy” tuck, or the like. In embodiments the surgery can comprise other types of medical procedures, for example appendix removal, organ transplant, and the like. In embodiments, methods comprise administering disclosed compositions in proximity to a wound.

Embodiments disclosed herein can reduce local muscular activity and thereby reduce the development of scars, for example scars resulting from trauma. For example, following a traumatic injury, disclosed embodiments can comprise administering disclosed compositions in proximity to trauma, for example a laceration or amputation.

Administration sites useful for practicing disclosed embodiments can comprise any area where muscle activity is to be reduced. For example, when employed in combination with facial cosmetic surgery procedures, disclosed embodiments can include administration to the glabellar complex, including the corrugator supercilli and the procerus; the obicularis oculi; the superolateral fibers of the obicularis oculi; the frontalis; the nasalis; the levator labii superioris aleque nasi; the obicularis oris; the masseter; the depressor anguli oris; and the platysma.

When employed in combination with other surgical procedures, disclosed embodiments can include administration to, for example, muscles of the face, arm, leg, torso, and the like.

Disclosed embodiments can comprise methods for preparing a surgical site prior to the procedure, in order to reduce muscle tension in the proximity of an incision.

Disclosed embodiments can promote the production of, for example, elastin, collagen, and the like. Disclosed embodiments can comprise methods of increasing the elasticity of the skin.

In embodiments, compositions disclosed herein can comprise fast-acting botulinum toxins, for example, BoNT/E.

In embodiments, compositions disclosed herein can comprise long-acting botulinum toxins, for example, BoNT/A.

In embodiments, compositions disclosed herein can comprise fast-recovery botulinum toxins, for example, BoNT/E.

In embodiments, compositions disclosed herein can comprise fast acting, fast-recovery botulinum toxins, for example, BoNT/E.

### Definitions

“Administration,” or “to administer” means the step of giving (i.e. administering) a pharmaceutical composition or active ingredient to a subject. The pharmaceutical compositions disclosed herein can be administered via a number of appropriate routs, however as described in the disclosed methods, the compositions are locally administered by e.g. intramuscular routes of administration, such as by injection or use of an implant.

“Botulinum toxin” or “botulinum neurotoxin” means a wild type neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the present invention. “Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Biocompatible” means that there is an insignificant inflammatory response at the site of implantation of an implant.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Entirely free” (“consisting of” terminology) means that within the detection range of the instrument or process being used, the substance cannot be detected or its presence cannot be confirmed.

“Essentially free” means that within the detection range of the instrument or process being used, only trace amounts of the substance can be detected.

“Fast-acting” as used herein refers to a botulinum toxin that produces effects in the patient more rapidly than those produced by, for example, BoNT/A. For example, the effects of a fast-acting botulinum toxin can be visible within 36 hours.

“Fast-recovery” as used herein refers to a botulinum toxin that whose effects diminish in the patient more rapidly than those produced by, for example, a BoNT/A. For example, the effects of a fast-recovery botulinum toxin can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months. However, the usual duration of an intramuscular injection of a botulinum neurotoxin type A is typically about 3 to 4 months.

“Intermediate-acting” as used herein refers to a botulinum toxin that produces effects more slowly that a fast-acting toxin.

“Neurotoxin” means a biologically active molecule with a specific affinity for a neuronal cell surface receptor. Neurotoxin includes Clostridial toxins both as pure toxin and as complexed with one to more non-toxin, toxin associated proteins.

“Patient” means a human or non-human subject receiving medical or veterinary care.

“Pharmaceutical composition” means a formulation in which an active ingredient can be a botulinum toxin. The word “formulation” means that there is at least one additional ingredient (such as, for example and not limited to, an albumin [such as a human serum albumin or a recombinant human albumin] and/or sodium chloride) in the pharmaceutical composition in addition to a botulinum neurotoxin active ingredient. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid or solid. A pharmaceutical composition can be animal-protein free.

“Substantially free” means present at a level of less than one percent by weight of a culture medium, fermentation medium, pharmaceutical composition or other material in which the weight percent of a substance is assessed.

“Supplemental administration” as used herein refers to a botulinum administration that follows an initial neurotoxin administration, for example to augment the effects of the initial administration.

“Therapeutic formulation” means a formulation that can be used to treat and thereby alleviate a disorder or a disease and/or symptom associated thereof, such as a disorder or a disease characterized by an activity of a peripheral muscle.

“Therapeutically effective amount” means the level, amount or concentration of an agent (e.g. such as a botulinum toxin or pharmaceutical composition comprising botulinum toxin) needed to treat a disease, disorder or condition without causing significant negative or adverse side effects.

“Toxin-naïve” means a patient who has not been administered a neurotoxin, for example a clostridial toxin, for example a botulinum type E neurotoxin.

“Treat,” “treating,” or “treatment” means an alleviation or a reduction (which includes some reduction, a significant reduction a near total reduction, and a total reduction), resolution or prevention (temporarily or permanently) of an disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active BoNT standardized to have equivalent neuromuscular blocking effect as a Unit of commercially available BoNT/A.

Neurotoxin Compositions

Embodiments disclosed herein comprise neurotoxin compositions, for example fast-acting neurotoxin compositions, for example BoNT/E compositions. Embodiments disclosed herein comprise neurotoxin compositions, for example long-acting neurotoxin compositions, for example BoNT/A compositions. Embodiments disclosed herein comprise combination neurotoxin compositions, for example comprising long-acting and fast-acting neurotoxin compositions, for example compositions comprising BoNT/A and BoNT/E. Embodiments disclosed herein can comprise multiple neurotoxins. For example, in embodiments disclosed compositions can comprise two types of neurotoxins, for example two types of botulinum neurotoxins, such as a fast-acting and a slower-acting neurotoxin, for example BoNT/E and BoNT/A. In embodiments, disclosed compositions can comprise a fragment of a botulinum neurotoxin, for example, a 50 kDa light chain (LC).

Such neurotoxins can be formulated in any pharmaceutically acceptable formulation in any pharmaceutically acceptable form. The neurotoxin can also be used in any pharmaceutically acceptable form supplied by any manufacturer.

Embodiments disclosed herein comprise neurotoxin compositions, for example fast-recovery neurotoxins. Such neurotoxins can be formulated in any pharmaceutically acceptable formulation in any pharmaceutically acceptable form. The neurotoxin can also be used in any pharmaceutically acceptable form supplied by any manufacturer.

The neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinant produced neurotoxin or a derivative or fragment thereof.

In embodiments, a disclosed BoNT/E composition has 40% amino acid homology compared with BoNT/A and they share the same basic domain structure consisting of 2 chains, a 100 kDa heavy chain (HC) and a 50 kDa light chain (LC), linked by a disulfide bond (Whelan 1992). The HC contains the receptor binding domain and the translocation domain while the LC contains the synaptosomal-associated protein (SNAP) enzymatic activity. The domain structure is the same structure shared by all botulinum neurotoxin serotypes.

In disclosed embodiments, the neurotoxin is formulated in unit dosage form; for example, it can be provided as a sterile solution in a vial or as a vial or sachet containing a lyophilized powder for reconstituting a suitable vehicle such as saline for injection.

In embodiments, the botulinum toxin is formulated in a solution containing saline and pasteurized human serum albumin, which stabilizes the toxin and minimizes loss through non-specific adsorption. The solution can comprise a buffer, for example a buffer with a PKa value between 6.0 and 8.0, high water solubility, minimal organic solubility, such as, for example, phosphate buffer, and other suitable types. The solution can be sterile filtered (0.2μ filter), filled into individual vials and then vacuum-dried to give a sterile lyophilized powder. In use, the powder can be reconstituted by the addition of sterile unpreserved normal saline (sodium chloride 0.9% for injection).

In an embodiment, BoNT/E is supplied in a sterile solution for injection with a 5-mL vial nominal concentration of 20 ng/mL in 0.03 M sodium phosphate, 0.12 M sodium chloride, and 1 mg/mL Human Serum Albumin (HSA), at pH 6.0.

In an embodiment, BoNT/E is supplied in a sterile solution for injection with a 5-mL vial nominal concentration of 10 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 5 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 1 ng/mL in 0.03 M sodium phosphate, 0.12 M sodium chloride, and 1 mg/mL HSA, at pH 6.0.

In an embodiment, BoNT/A is supplied in a sterile solution for injection with a 5-mL vial nominal concentration of 20 ng/mL in 0.03 M sodium phosphate, 0.12 M sodium chloride, and 1 mg/mL Human Serum Albumin (HSA), at pH 6.0.

In an embodiment, BoNT/A is supplied in a sterile solution for injection with a 5-mL vial nominal concentration of 10 ng/mL in 0.03 M sodium phosphate, 0.12 M sodium chloride, and 1 mg/mL HSA, at pH 6.0.

In an embodiment, BoNT/A is supplied in a sterile solution for injection with a 5-mL vial nominal concentration of 5 ng/mL in 0.03 M sodium phosphate, 0.12 M sodium chloride, and 1 mg/mL HSA, at pH 6.0.

In an embodiment, BoNT/A is supplied in a sterile solution for injection with a 5-mL vial nominal concentration of 1 ng/mL in 0.03 M sodium phosphate, 0.12 M sodium chloride, and 1 mg/mL HSA, at pH 6.0.

In an embodiment, the combination of botulinum neurotoxins is supplied in a sterile solution for injection with a 5-mL vial nominal concentration of 20 ng/mL in 0.03 M sodium phosphate, 0.12 M sodium chloride, and 1 mg/mL Human Serum Albumin (HSA), at pH 6.0.

In an embodiment, the combination of botulinum neurotoxins is supplied in a sterile solution for injection with a 5-mL vial nominal concentration of 10 ng/mL in 0.03 M sodium phosphate, 0.12 M sodium chloride, and 1 mg/mL HSA, at pH 6.0.

In an embodiment, the combination of botulinum neurotoxins is supplied in a sterile solution for injection with a 5-mL vial nominal concentration of 5 ng/mL in 0.03 M sodium phosphate, 0.12 M sodium chloride, and 1 mg/mL HSA, at pH 6.0.

In an embodiment, the combination of botulinum neurotoxins is supplied in a sterile solution for injection with a 5-mL vial nominal concentration of 1 ng/mL in 0.03 M sodium phosphate, 0.12 M sodium chloride, and 1 mg/mL HSA, at pH 6.0.

Disclosed compositions can also comprise agents that modulate dopamine receptors, such as antipsychotics, norepinephrine receptors, and/or serotonin receptors. The compositions may also include agents that affect ion flux through voltage gated calcium channels, potassium channels, and/or sodium channels. Thus, the compositions used in disclosed embodiments may include one or more neurotoxins, such as botulinum toxins, in addition to ion channel receptor modulators that may reduce neurotransmission.

Disclosed compositions can comprise at least one opioid. For example, disclosed embodiments can comprise codeine, alfentanil, fentanyl, remifentanil, sufentanil, buprenorphine, butorphanol, diacetyl morphine, (diamorphine), hydromorphone, levorphanol meperidine, also called pethidine in the UK, New Zealand, Australia and other countries, methadone, hydrocodone, morphine, nalbuphine, naltrexone, oxycodone, oxymorphone, pentazocine, meperidine, morphine, oripavine, pseudomorphine, thebaine, 14-hydroxymorphine, 2,4-dinitrophenylmorphine, 6-methyldihydromorphine, 6-methylenedihydrodesoxymorphine, 6-acetyldihydromorphine, azidomorphine, chlornaltrexamine, chloroxymorphamine, desomorphine (dihydrodesoxymorphine), dihydromorphine, ethyldihydromorphine, hydromorphinol, methyldesorphine, morphine methylbromide, N-phenethylnordesomorphine, N-phenethylnormorphine, 6-nicotinoyldihydromorphine (metabolite of nicodicodeine), RAM-378, acetylpropionylmorphine, 3,6-dibutanoylmorphine, diacetyldihydromorphine (dihydroheroin, acetylmorphinol), dibutyrylmorphine, dibenzoylmorphine, diformylmorphine, dipropanoylmorphine, Heroin (diacetylmorphine), nicomorphine, 14-cinnamoyloxycodeinone, 14-Ethoxymetopon, 14-methoxymetopon, 14-phenylpropoxymetopon, 3-acetyloxymorphone, 3,14-diacetyloxymorphone, 7-spiroindanyloxymorphone, 8,14-dihydroxydihydromorphinone, acetylcodone, acetylmorphone, α-hydrocodol, benzhydrocodone, bromoisopropropyldihydromorphinone, codeinone, codol, codoxime, conorfone (codorphone), IBNtxA, thebacon (acetyldihydrocodeinone, dihydrocodeinone enol acetate), hydromorphone, hydroxycodeine, metopon, morphenol, morphinone, morphol, N-phenethyl-14-ethoxymetopon, noroxymorphone, oxycodone, oxymorphol, oxymorphone, pentamorphone, semorphone, 5,9 alpha-diethyl-2-hydroxybenzomorphan (5,9-DEHB), 8-carboxamidocyclazocine (8-CAC), alazocine, anazocine, bremazocine, butinazocine, carbazocine, cogazocine, cyclazocine, dezocine, eptazocine, etazocine, ethylketazocine, fedotozine, fluorophen, gemazocine, ibazocine, ketazocine, metazocine, moxazocine, pentazocine, phenazocine, quadazocine, SKF-10047, thiazocine, tonazocine, volazocine, zenazocine, 4-fluoropethidine, allylnorpethidine, anileridine, benzethidine, carperidine, difenoxin, diphenoxylate, etoxeridine (carbetidine), furethidine, hydroxypethidine (bemidone), morpheridine, meperidine-N-oxide, oxpheneridine (carbamethidine), pethidine (meperidine), pethidine intermediate A, pethidine intermediate B (norpethidine), pethidine intermediate C (pethidinic acid), pheneridine, phenoperidine, piminodine, properidine (ipropethidine), sameridine, dextromethadone, dipipanone, isomethadone, levoisomethadone, levomethadone, methadone intermediate, normethadone, norpipanone, phenadoxone (heptazone), 3-allylfentanyl, 3-methylfentanyl, 3-methylthiofentanyl, 4-Phenylfentanyl, alfentanil, α-methylacetylfentanyl, α-methylfentanyl, α-methylthiofentanyl, benzylfentanyl, hydroxyfentanyl, β-hydroxythiofentanyl, β-methylfentanyl, brifentanil, butyrfentanyl, carfentanil, lofentanil, N-methylcarfentanil, mirfentanil, ocfentanil, ohmefentanyl, parafluorofentanyl, phenaridine, R-30490, remifentanil, sufentanil, thenylfentanyl, thiofentanyl, trefentanil, adrenorphin, amidorphin, biphalin, casokefamide, casomorphins, cytochrophin-4, DALDA (Tyr-D-Arg-Phe-Lys-NH₂), deltorphin A, deltorphin I, deltorphin II, deprolorphin, dermorphin, DPDPE, frakefamide, gliadorphin, gluten exorphinss, hemorphin-4, metkefamide, morphiceptin, nociceptin, octreotide, opiorphin, rubiscolin, soymorphins, spinorphin, TRIMU 5, tynorphin, valorphin, zyklophin, analogs thereof, or combinations thereof.

Disclosed embodiments can comprise an anesthetic. For example, disclosed methods and compositions can comprise general, regional, or local anesthetics, or combinations thereof, or the like.

Extended Duration/Controlled Release

A controlled release system can be used in the embodiments described herein to deliver neurotoxin compositions in vivo at a predetermined rate over a specific time period. Generally, release rates are determined by the design of the system, and can be largely independent of environmental conditions such as pH. Controlled release systems which can deliver a drug over a period of several years are known. Contrarily, sustained release systems typically deliver drug in 24 hours or less and environmental factors can influence the release rate. Thus, the release rate of a neurotoxin from an implanted controlled release system (an “implant”) is a function of the physiochemical properties of the carrier implant material and of the drug itself. Typically, the implant is made of an inert material which elicits little or no host response.

A controlled release system as described herein can be comprised of a neurotoxin, for example, BoNT/E, incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

A membrane or reservoir implant depends upon the diffusion of a neurotoxin across a polymer membrane. A matrix implant is comprised of a polymeric matrix in which the neurotoxin is uniformly distributed. Swelling-controlled release systems are usually based on hydrophilic, glassy polymers which undergo swelling in the presence of biological fluids or in the presence of certain environmental stimuli.

Implants may be prepared by mixing a desired amount of a stabilized neurotoxin into a solution of a suitable polymer dissolved in methylene chloride. The solution may be prepared at room temperature. The solution can then be transferred to a Petri dish and the methylene chloride evaporated in a vacuum desiccator. Depending upon the implant size desired and hence the amount of incorporated neurotoxin, a suitable amount of the dried neurotoxin incorporating implant is compressed at about 8000 p.s.i. for 5 seconds or at 3000 p.s.i. for 17 seconds in a mold to form implant discs encapsulating the neurotoxin.

Preferably, the implant material used is substantially non-toxic, non-carcinogenic, and non-immunogenic. Suitable implant materials include polymers, such as poly(2-hydroxy ethyl methacrylate) (β-HEMA), poly(N-vinyl pyrrolidone) (β-NVP)+, poly(vinyl alcohol) (PVA), poly(acrylic acid) (PM), polydimethyl siloxanes (PDMS), ethylene-vinyl acetate (EVAc) copolymers, hyaluronic acid, polyvinylpyrrolidone/methylacrylate copolymers, polymethylmethacrylate (PMMA), poly(lactic acid) (PLA), poly(glycolic acid) (PGA), polyanhydrides, poly(ortho esters), collagen and cellulosic derivatives and bioceramics, such as hydroxyapatite (HPA), tricalcium phosphate (TCP), and aliminocalcium phosphate (ALCAP). Lactic acid, glycolic acid and collagen can be used to make biodegradable implants.

In embodiments, the implant material can be biodegradable or bioerodible. An advantage of a bioerodible implant is that it does not need to be removed from the patient. A bioerodible implant can be based upon either a membrane or matrix release of the bioactive substance. Biodegradable microspheres prepared from PLA-PGA are known for subcutaneous or intramuscular administration.

Also disclosed herein are thermo-reversible, thermoplastic, pharmaceutical compositions that include at least one biologically active botulinum toxin and a thermo-reversible, thermoplastic poloxamer, wherein the poloxamer stabilizes the botulinum toxin and is a gel at room temperature (e.g. the temperature of an enclosed space at which human beings are usually accustomed, e.g. from about 17° C. to about 25° C. Before administration, the pharmaceutical composition is cooled below room temperature to reduce its viscosity (liquefy) the pharmaceutical composition and is thereafter drawn into a syringe and injected into the patient, where the thermoplastic pharmaceutical composition gels to deliver therapeutic amounts of the botulinum toxin are released from the composition in vivo for at least 1 week after administration. In particular embodiments, the thermoplastic poloxamer is a poloxamer 407 and is present at a concentration of about 15 wt % to about 25 wt % of the pharmaceutical composition.

In embodiments, the thermo-reversible, thermoplastic poloxamer such as poloxamer 407 at about 20% wt (and hence the thermoplastic, pharmaceutical composition) can have a first viscosity at a first temperature (e.g. from about 0 centipoise (cP) at about 0 to about 16° C.), have its temperature raised to increase its viscosity to a second viscosity that is higher relative to the first viscosity (e.g. from about 50 cP to about 6000 cP at about 18 to about 22° C.), and then is reversible, e.g. lowering its temperature, decreasing its viscosity relative to the second viscosity, for example. A change in weight % of poloxamer 407 in a composition will alter its viscosity/temperature profile.

Embodiments can include in situ-forming, injectable hydrogels, for example thermally-responsive hydrogels. Embodiments disclosed herein can comprise a thermal gel, for example an amine-functionalized ABA block copolymer, poly(ethylene glycol)-poly(serinol hexamethylene urethane), or ESHU.

Methods of Use

Methods disclosed herein can comprise selecting a patient for treatment. For example, selecting a patient who could benefit from administration or performance of the methods and/or compositions disclosed herein.

Disclosed embodiments comprise administration of a botulinum toxin concurrently with administration of an opioid. Disclosed embodiments comprise administration of a botulinum toxin concurrently instead of administration of an opioid. Disclosed embodiments comprise administration of a botulinum toxin concurrently with an opioid withdrawal treatment. In embodiments, the patient has not been previously treated with an opioid.

Embodiments comprise administration of a fast-acting neurotoxin prior to administration of an opioid. In embodiments, the administration is performed, for example, within 48 hours before administration of an opioid, within 47 hours before administration of an opioid, within 46 hours before administration of an opioid, within 45 hours before administration of an opioid, within 44 hours before administration of an opioid, within 43 hours before administration of an opioid, within 42 hours before administration of an opioid, within 41 hours before administration of an opioid, within 40 hours before administration of an opioid, within 39 hours before administration of an opioid, within 38 hours before administration of an opioid, within 37 hours before administration of an opioid, within 36 hours before administration of an opioid, within 35 hours before administration of an opioid, within 34 hours before administration of an opioid, within 33 hours before administration of an opioid, within 32 hours before administration of an opioid, within 31 hours before administration of an opioid, within 30 hours before administration of an opioid, within 29 hours before administration of an opioid, within 28 hours before administration of an opioid, within 27 hours before administration of an opioid, within 26 hours before administration of an opioid, within 25 hours before administration of an opioid, within 24 hours before administration of an opioid, within 23 hours before administration of an opioid, within 22 hours before administration of an opioid, within 21 hours before administration of an opioid, within 20 hours before administration of an opioid, within 19 hours before administration of an opioid, within 18 hours before administration of an opioid, within 17 hours before administration of an opioid, within 16 hours before administration of an opioid, within 15 hours before administration of an opioid, within 14 hours before administration of an opioid, within 13 hours before administration of an opioid, within 12 hours before administration of an opioid, within 11 hours before administration of an opioid, within 10 hours before administration of an opioid, within 9 hours before administration of an opioid, within 8 hours before administration of an opioid, within 7 hours before administration of an opioid, within 6 hours before administration of an opioid, within 5 hours before administration of an opioid, within 4 hours before administration of an opioid, within 3 hours before administration of an opioid, within 2 hours before administration of an opioid, within 60 minutes before administration of an opioid, within 50 minutes before administration of an opioid, within 40 minutes before administration of an opioid, within 30 minutes before administration of an opioid, within 20 minutes before administration of an opioid, within 10 minutes before administration of an opioid, within 5 minutes before administration of an opioid, within 2 minutes before administration of an opioid, or the like.

Embodiments comprise administration of a fast-acting neurotoxin prior to administration of an opioid. In embodiments, the administration is performed, for example, within 48 hours or less before administration of an opioid, within 47 hours or less before administration of an opioid, within 46 hours or less before administration of an opioid, within 45 hours or less before administration of an opioid, within 44 hours or less before administration of an opioid, within 43 hours or less before administration of an opioid, within 42 hours or less before t administration of an opioid, within 41 hours or less before administration of an opioid, within 40 hours or less before administration of an opioid, within 39 hours or less before administration of an opioid, within 38 hours or less before administration of an opioid, within 37 hours or less before administration of an opioid, within 36 hours or less before administration of an opioid, within 35 hours or less before administration of an opioid, within 34 hours or less before administration of an opioid, within 33 hours or less before administration of an opioid, within 32 hours or less before administration of an opioid, within 31 hours or less before administration of an opioid, within 30 hours or less before administration of an opioid, within 29 hours or less before administration of an opioid, within 28 hours or less before administration of an opioid, within 27 hours or less before administration of an opioid, within 26 hours or less before administration of an opioid, within 25 hours or less before administration of an opioid, within 24 hours or less before administration of an opioid, within 23 hours or less before administration of an opioid, within 22 hours or less before administration of an opioid, within 21 hours or less before administration of an opioid, within 20 hours or less before administration of an opioid, within 19 hours or less before administration of an opioid, within 18 hours or less before administration of an opioid, within 17 hours or less before administration of an opioid, within 16 hours or less before administration of an opioid, within 15 hours or less before administration of an opioid, within 14 hours or less before administration of an opioid, within 13 hours or less before administration of an opioid, within 12 hours or less before administration of an opioid, within 11 hours or less before administration of an opioid, within 10 hours or less before administration of an opioid, within 9 hours or less before administration of an opioid, within 8 hours or less before administration of an opioid, within 7 hours or less before administration of an opioid, within 6 hours or less before administration of an opioid, within 5 hours or less before administration of an opioid, within 4 hours or less before administration of an opioid, within 3 hours or less before administration of an opioid, within 2 hours or less before administration of an opioid, within 60 minutes or less before administration of an opioid, within 50 minutes or less before administration of an opioid, within 40 minutes or less before administration of an opioid, within 30 minutes or less before administration of an opioid, within 20 minutes or less before administration of an opioid, within 10 minutes or less before administration of an opioid, within 5 minutes or less before administration of an opioid, within 2 minutes or less before administration of an opioid, or the like.

In embodiments, administration of the fast-acting neurotoxin is performed concurrently with a surgical procedure. In embodiments, administration of the opioid is performed concurrently with a surgical procedure.

In embodiments, administration of the fast-acting neurotoxin is performed after administration of an opioid. For example, administration can be performed, within 1 minute after administration of an opioid, within 2 minutes after administration of an opioid, within 3 minutes after administration of an opioid, within 4 minutes after administration of an opioid, within 5 minutes after administration of an opioid, within 6 minutes after administration of an opioid, within 7 minutes after administration of an opioid, within 8 minutes after administration of an opioid, within 9 minutes after administration of an opioid, within 10 minutes after administration of an opioid, within 20 minutes after administration of an opioid, within 30 minutes after administration of an opioid, within 40 minutes after administration of an opioid, within 50 minutes after administration of an opioid, within 60 minutes after administration of an opioid, within 90 minutes after administration of an opioid, within 120 minutes after administration of an opioid, within 180 minutes after administration of an opioid, within 240 minutes after administration of an opioid, within 300 minutes after administration of an opioid, or the like.

Disclosed embodiments comprise administration of a neurotoxin in combination with administration of an anesthetic. For example, embodiments comprise administration of a fast-acting neurotoxin prior to administration of an anesthetic. In embodiments, the administration is performed, for example, within 48 hours before administration of an anesthetic, within 47 hours before administration of an anesthetic, within 46 hours before administration of an anesthetic, within 45 hours before administration of an anesthetic, within 44 hours before administration of an anesthetic, within 43 hours before administration of an anesthetic, within 42 hours before administration of an anesthetic, within 41 hours before administration of an anesthetic, within 40 hours before administration of an anesthetic, within 39 hours before administration of an anesthetic, within 38 hours before administration of an anesthetic, within 37 hours before administration of an anesthetic, within 36 hours before administration of an anesthetic, within 35 hours before administration of an anesthetic, within 34 hours before administration of an anesthetic, within 33 hours before administration of an anesthetic, within 32 hours before administration of an anesthetic, within 31 hours before administration of an anesthetic, within 30 hours before administration of an anesthetic, within 29 hours before administration of an anesthetic, within 28 hours before administration of an anesthetic, within 27 hours before administration of an anesthetic, within 26 hours before administration of an anesthetic, within 25 hours before administration of an anesthetic, within 24 hours before administration of an anesthetic, within 23 hours before administration of an anesthetic, within 22 hours before administration of an anesthetic, within 21 hours before administration of an anesthetic, within 20 hours before administration of an anesthetic, within 19 hours before administration of an anesthetic, within 18 hours before administration of an anesthetic, within 17 hours before administration of an anesthetic, within 16 hours before administration of an anesthetic, within 15 hours before administration of an anesthetic, within 14 hours before administration of an anesthetic, within 13 hours before administration of an anesthetic, within 12 hours before administration of an anesthetic, within 11 hours before administration of an anesthetic, within 10 hours before administration of an anesthetic, within 9 hours before administration of an anesthetic, within 8 hours before administration of an anesthetic, within 7 hours before administration of an anesthetic, within 6 hours before administration of an anesthetic, within 5 hours before administration of an anesthetic, within 4 hours before administration of an anesthetic, within 3 hours before administration of an anesthetic, within 2 hours before administration of an anesthetic, within 60 minutes before administration of an anesthetic, within 50 minutes before administration of an anesthetic, within 40 minutes before administration of an anesthetic, within 30 minutes before administration of an anesthetic, within 20 minutes before administration of an anesthetic, within 10 minutes before administration of an anesthetic, within 5 minutes before administration of an anesthetic, within 2 minutes before administration of an anesthetic, or the like.

Embodiments comprise administration of a fast-acting neurotoxin prior to administration of an anesthetic. In embodiments, the administration is performed, for example, within 48 hours or less before administration of an anesthetic, within 47 hours or less before administration of an anesthetic, within 46 hours or less before administration of an anesthetic, within 45 hours or less before administration of an anesthetic, within 44 hours or less before administration of an anesthetic, within 43 hours or less before administration of an anesthetic, within 42 hours or less before t administration of an anesthetic, within 41 hours or less before administration of an anesthetic, within 40 hours or less before administration of an anesthetic, within 39 hours or less before administration of an anesthetic, within 38 hours or less before administration of an anesthetic, within 37 hours or less before administration of an anesthetic, within 36 hours or less before administration of an anesthetic, within 35 hours or less before administration of an anesthetic, within 34 hours or less before administration of an anesthetic, within 33 hours or less before administration of an anesthetic, within 32 hours or less before administration of an anesthetic, within 31 hours or less before administration of an anesthetic, within 30 hours or less before administration of an anesthetic, within 29 hours or less before administration of an anesthetic, within 28 hours or less before administration of an anesthetic, within 27 hours or less before administration of an anesthetic, within 26 hours or less before administration of an anesthetic, within 25 hours or less before administration of an anesthetic, within 24 hours or less before administration of an anesthetic, within 23 hours or less before administration of an anesthetic, within 22 hours or less before administration of an anesthetic, within 21 hours or less before administration of an anesthetic, within 20 hours or less before administration of an anesthetic, within 19 hours or less before administration of an anesthetic, within 18 hours or less before administration of an anesthetic, within 17 hours or less before administration of an anesthetic, within 16 hours or less before administration of an anesthetic, within 15 hours or less before administration of an anesthetic, within 14 hours or less before administration of an anesthetic, within 13 hours or less before administration of an anesthetic, within 12 hours or less before administration of an anesthetic, within 11 hours or less before administration of an anesthetic, within 10 hours or less before administration of an anesthetic, within 9 hours or less before administration of an anesthetic, within 8 hours or less before administration of an anesthetic, within 7 hours or less before administration of an anesthetic, within 6 hours or less before administration of an anesthetic, within 5 hours or less before administration of an anesthetic, within 4 hours or less before administration of an anesthetic, within 3 hours or less before administration of an anesthetic, within 2 hours or less before administration of an anesthetic, within 60 minutes or less before administration of an anesthetic, within 50 minutes or less before administration of an anesthetic, within 40 minutes or less before administration of an anesthetic, within 30 minutes or less before administration of an anesthetic, within 20 minutes or less before administration of an anesthetic, within 10 minutes or less before administration of an anesthetic, within 5 minutes or less before administration of an anesthetic, within 2 minutes or less before administration of an anesthetic, or the like.

In embodiments, administration of the fast-acting neurotoxin is performed concurrently with a surgical procedure. In embodiments, administration of the anesthetic is performed concurrently with a surgical procedure.

In embodiments, administration of the fast-acting neurotoxin is performed after administration of an anesthetic. For example, administration can be performed, within 1 minute after administration of an anesthetic, within 2 minutes after administration of an anesthetic, within 3 minutes after administration of an anesthetic, within 4 minutes after administration of an anesthetic, within 5 minutes after administration of an anesthetic, within 6 minutes after administration of an anesthetic, within 7 minutes after administration of an anesthetic, within 8 minutes after administration of an anesthetic, within 9 minutes after administration of an anesthetic, within 10 minutes after administration of an anesthetic, within 20 minutes after administration of an anesthetic, within 30 minutes after administration of an anesthetic, within 40 minutes after administration of an anesthetic, within 50 minutes after administration of an anesthetic, within 60 minutes after administration of an anesthetic, within 90 minutes after administration of an anesthetic, within 120 minutes after administration of an anesthetic, within 180 minutes after administration of an anesthetic, within 240 minutes after administration of an anesthetic, within 300 minutes after administration of an anesthetic, or the like.

Methods disclosed herein can comprise administering to a patient in need thereof an effective amount of a combination of botulinum neurotoxins. The methods can also include the step of identifying that the subject is in need of treatment of diseases or disorders associated with botulinum toxin therapy. For example, an effective amount of BoNT/A and BoNT/E can be administered to the identified subject. The identification can be in the judgment of a subject or a health professional and can be subjective (e.g., opinion) or objective (e.g., measurable by a test or a diagnostic method). The methods delineated herein can further include the step of assessing or identifying the effectiveness of the treatment or prevention regimen in the subject by assessing the presence, absence, increase, or decrease of a marker. Such assessment methodologies are know in the art and can be performed by commercial diagnostic or medical organizations, laboratories, clinics, hospitals and the like. The methods can further include the step of taking a sample from the subject and analyzing that sample. The sample can be a sampling of cells, genetic material, tissue, or fluid (e.g., blood, plasma, sputum, etc.) sample. The methods can further include the step of reporting the results of such analyzing to the subject or other health care professional.

In certain embodiments, BoNT/A to BoNT/E are administered in approximately equal amounts, i.e., in approximately a 1:1 ratio, based on weight or molar amounts. In other embodiments, the ratio of BoNT/A to BoNT/E administered is approximately 0.001:1, 0.005:1, 0.01:1, 0.05:1, 0.1:1, 0.2:1, 0.3:1, 0.4:1, 0.5:1, 0.6:1, 0.7:1, 0.8:1, 0.9:1, 1.1:1, 1.2:1, 1.3:1, 1.4:1, 1.5:1, 1.6:1, 1.7:1, 1.8:1, 1.9:1, 2:1, 3:1, 4:1, 5:1, 10:1, 100:1, 300:1, 500:1, 1000:1 or 10,000:1. In other embodiments, the ratio of BoNT/E to BoNT/A administered is approximately 0.001:1, 0.005:1, 0.01:1, 0.05:1, 0.1:1, 0.2:1, 0.3:1, 0.4:1, 0.5:1, 0.6:1, 0.7:1, 0.8:1, 0.9:1, 1.1:1, 1.2:1, 1.3:1, 1.4:1, 1.5:1, 1.6:1, 1.7:1, 1.8:1, 1.9:1, 2:1, 3:1, 4:1, 5:1, 10:1, 100:1, 300:1, 500:1, 1000:1 or 10,000:1.

In one embodiment, the botulinum toxins are in an admixture. In one embodiment, approximately equal amounts of BoNT/A and BoNT/E are administered simultaneously. As used herein “simultaneously” means that the two neurotoxins are administered at approximately the same time, e.g., in a mixture or as multiple administration. Alternatively, simultaneously can mean the administration of one botulinum neurotoxin prior to the administration of the second botulinum neurotoxin such that the second botulinum neurotoxin is administered prior to the first botulinum neurotoxin achieving full effect, e.g., preferably long before the first botulinum toxin achieves full effect. In preferred embodiments, the second botulinum toxin is administered within 120 hours, 96 hours, 72 hours, 48 hours, 24 hours, 12 hours, 6 hours, 4 hours, 3 hours, 2 hours, 1 hour, 45 minutes, 30 minutes, 20 minutes, 15 minutes, 10 minutes, 5 minutes, 4 minutes, 3 minutes, 2 minutes, or 1 minute of the first botulinum toxin. In the methods disclosed herein, either neurotoxin can be administered first. In further methods disclosed herein, the fast-acting neurotoxin is administered first. In further methods disclosed herein, the slower-acting neurotoxin is administered first. In further methods disclosed herein, the fast-recovery neurotoxin is administered first. In further methods disclosed herein, the slower-recovery neurotoxin is administered first.

In embodiments, the patient has not been previously treated with a neurotoxin, for example a clostridial toxin, such as a botulinum toxin. In embodiments, the patient has not been previously treated with a fast-acting neurotoxin. In embodiments, the patient has not been previously treated with a fast-recovery neurotoxin. In embodiments, the patient has not been previously treated with a fast-acting, fast-recovery neurotoxin.

Embodiments comprise administration of a fast-acting neurotoxin in conjunction with a surgical procedure, for example prior to a surgical procedure. In embodiments, the administration is performed, for example, within 48 hours before the procedure, within 47 hours before the procedure, within 46 hours before the procedure, within 45 hours before the procedure, within 44 hours before the procedure, within 43 hours before the procedure, within 42 hours before the procedure, within 41 hours before the procedure, within 40 hours before the procedure, within 39 hours before the procedure, within 38 hours before the procedure, within 37 hours before the procedure, within 36 hours before the procedure, within 35 hours before the procedure, within 34 hours before the procedure, within 33 hours before the procedure, within 32 hours before the procedure, within 31 hours before the procedure, within 30 hours before the procedure, within 29 hours before the procedure, within 28 hours before the procedure, within 27 hours before the procedure, within 26 hours before the procedure, within 25 hours before the procedure, within 24 hours before the procedure, within 23 hours before the procedure, within 22 hours before the procedure, within 21 hours before the procedure, within 20 hours before the procedure, within 19 hours before the procedure, within 18 hours before the procedure, within 17 hours before the procedure, within 16 hours before the procedure, within 15 hours before the procedure, within 14 hours before the procedure, within 13 hours before the procedure, within 12 hours before the procedure, within 11 hours before the procedure, within 10 hours before the procedure, within 9 hours before the procedure, within 8 hours before the procedure, within 7 hours before the procedure, within 6 hours before the procedure, within 5 hours before the procedure, within 4 hours before the procedure, within 3 hours before the procedure, within 2 hours before the procedure, within 60 minutes before the procedure, within 50 minutes before the procedure, within 40 minutes before the procedure, within 30 minutes before the procedure, within 20 minutes before the procedure, within 10 minutes before the procedure, within 5 minutes before the procedure, within 2 minutes before the procedure, or the like.

Ultimately, however, both the quantity of toxin administered and the frequency of its administration will be at the discretion of the physician responsible for the treatment and will be commensurate with questions of safety and the effects produced by the toxin.

In specific embodiments, the botulinum toxins are administered intramuscularly, subcutaneously, or transdermally.

In the case of intramuscular administration, before injecting any muscle group, careful consideration is given to the anatomy of the muscle group, the aim being to inject the area with the highest concentration of neuromuscular junctions, if known. Before injecting the muscle, the position of the needle in the muscle can be confirmed by putting the muscle through its range of motion and observing the resultant motion of the needle end. General anesthesia, local anesthesia and sedation are used according to the age of the patient, the number of sites to be injected, and the particular needs of the patient. More than one injection and/or sites of injection may be necessary to achieve the desired result. Also, some injections, depending on the muscle to be injected, may require the use of fine, hollow, Teflon®-coated needles, guided by electromyography.

Administration Devices

Disclosed fast-acting neurotoxin compositions can be administered using a needle or a needleless device. In certain embodiments, the method comprises subdermally injecting the composition in the individual. For example, administration may comprise injecting the composition through a needle no greater than about 30 gauge. In certain embodiments, the method comprises administering a composition comprising a botulinum toxin type E.

Injection of the compositions can be carried out by syringe, catheters, needles and other means for injecting. The injection can be performed on any area of the mammal's body that is in need of treatment, including, but not limited to, face, neck, torso, arms, hands, legs, and feet. The injection can be into any position in the specific area such as epidermis, dermis, fat, muscle, or subcutaneous layer.

The frequency and the amount of injection under the disclosed methods can be determined based on the nature and location of the particular cosmetic irregularity being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. Such determinations are routine to one of ordinary skill in the art. For example, the route and dosage for administration of a Clostridial neurotoxin according to the present disclosed invention can be selected based upon criteria such as the solubility characteristics of the neurotoxin chosen as well as the intensity and scope of the cosmetic condition being treated.

Administration devices suitable for disclosed embodiments comprising on-site mixed or pre-mixed compositions can include a hypodermic needle, for example a sterile, disposable hypodermic needle.

Compositions disclosed herein can comprise a mix of botulinum neurotoxins. The compositions can be on-site mixed, in-device mixed, or premixed.

Embodiments comprising in-device mixed compositions can comprise multiple chambers, such that each botulinum neurotoxin occupies a separate chamber and is then mixed prior to administration, for example via a hypodermic needle. In certain embodiments, the device comprises input means wherein the practitioner can adjust the device to administer a ratio of one neurotoxin to another. In certain embodiments, the device comprises sampling ports for aseptic removal of neurotoxins. Certain embodiments comprise kits comprising neurotoxins, means for administration, and instructions for use.

Certain embodiments comprise at least two chambers, wherein a first chamber comprises a first botulinum neurotoxin solution, and a second chamber comprises a second botulinum neurotoxin solution. In certain embodiments, disclosed devices can comprise at least three chambers. In still further embodiments, a third chamber comprises a solution comprising one or more sequestering agents.

In certain embodiments, devices disclosed herein comprise one or more barriers or seals or septa separating the chambers. In embodiments, the barriers or seals or septa may be broken or breached or ruptured or punctured or permeabilized or perforated to allow the contents of each chamber to mix. In still further embodiments, the devices described herein comprise one or more barriers, septa or seals which may be rendered ineffective in preventing mixing of the solutions and compositions described herein. In certain embodiments of the invention, the devices described herein comprise one or more barriers or seals or septa, wherein said barriers or seals or septa comprise a membrane.

Embodiments include a pre-filled, ready-to-use, disposable syringe wherein two different neurotoxins, for example BoNT/A and BoNT/E, are sealed in two separate telescoping syringe barrels. In another embodiment, a lyophilized neurotoxin or neurotoxin combination is sealed in an outer barrel adjacent to the nozzle section by means of a pierceable stopper and the diluent is sealed in an inner barrel between another pierceable stopper and a plunger stopper. A double-pointed cannula is positioned between the two pierceable stoppers by means of a telescoping guide arrangement. Movement of the plunger stopper inwardly in the inner barrel initially effects a piercing of both pierceable stoppers and intermixing of the lyophilized neurotoxin or neurotoxin combination with the diluent. Continued movement of the plunger stopper will expel the mixed neurotoxins from the syringe.

Embodiments include two-chamber syringes for mixing a lyophilized neurotoxin or neurotoxin combination with a diluent and then injecting the mixed ingredients into a patient. The syringe may include a vial formed with a single glass cylinder closed at one end by a plunger and at its other end by a pierceable diaphragm. An intermediate pierceable diaphragm divides the cylinder into upper and lower chambers, and is locked against axial movement relative to the cylinder. A powdered medicament is provided in the upper chamber and a diluent is provided in the lower chamber. The ingredients are mixed by inserting the vial into a cup-shaped holder having a hollow, pointed needle extending from the base of the holder. Axial pressure on the vial causes the pointed end of the needle to sequentially pierce the end and intermediate diaphragms, and to cause the diluent to flow into an opening in the sidewall of the needle, through the needle, and then into the upper chamber from the pointed needle end. The thus-mixed ingredients are dispensed, for example, by applying axial pressure to the plunger, or by drawing metered amounts into the medicament pressurizing chamber of a needle-less, hypojet injector.

Further embodiments comprise a wet-dry syringe for combining and mixing a liquid botulinum neurotoxin composition and a solid botulinum neurotoxin composition or at least two dissimilar liquid botulinum neurotoxin compositions prior to the application thereof to a patient. Embodiments can comprise a first vial having a liquid botulinum neurotoxin composition or a solid botulinum neurotoxin composition disposed between a pair of identical vial seals.

Further embodiments include a multiple-chamber automatic injector. An automatic injector is disclosed having at least two chambers containing different botulinum neurotoxins separated by an impermeable membrane. A lance is movable independently of a plunger to cut or pierce the membrane before a spring-loaded drive member for the plunger is released to drive a needle out of the body of the injector and discharge the combination of different botulinum neurotoxins through the needle.

Disclosed embodiments comprise an automatic two-chamber injector. The injector comprises a barrel having a first end with a receiving portion for an injection needle, said portion being sealed prior to use, and a second end with a displaceable plunger. The barrel comprises two chambers separated by a migration-proof membrane, said membrane being adapted to rupture when the plunger is displaced towards the first end of the barrel. Also disclosed is a method for mixing and injecting a botulinum neurotoxin solution by means of an automatic two-chamber injector and to a cartridge for a two-chamber injector.

Further embodiments comprise a multiple chamber automatic injector having at least two chambers containing different botulinum neurotoxin compositions separated by an impermeable membrane. A lance is movable independently of a plunger to cut or pierce the membrane before a spring-loaded drive member for the plunger is released to drive a needle out of the body of the injector and discharge the mixed compositions through the needle.

Before administering compositions disclosed herein, careful consideration is given to the anatomy of the treatment site. For example, in embodiments, the therapeutic goal is to inject the area with the highest concentration of neuromuscular junctions, if known. For example, in the case of intramuscular administration, before injecting the muscle the position of the needle in the muscle can be confirmed by putting the muscle through its range of motion and observing the resultant motion of the needle end. General anesthesia, local anesthesia and sedation are used according to the age of the patient, the number of sites to be injected, and the particular needs of the patient. More than one injection and/or sites of injection may be necessary to achieve the desired result. Also, some injections, depending on the muscle to be injected, may require the use of fine, hollow, TEFLON®-coated needles, guided by electromyography.

Administration of disclosed compositions can comprise, for example, injection, into or in the vicinity of one or more of the following skeletal muscles, for example, the occipitofrontalis, nasalis, orbicularis oris, depressor anguli oris, platysma, sternohyoid, serratus anterior, rectus abdominis, external oblique, tensor fasciae latae, brachioradialis, Iliacus, psoas major, pectineus, adductor longus, sartorius, gracillis, vastus lateralis, rectus femoris, vastus medialis, tendon of quadriceps femoris, patella, gastroctnemius, soleus, tibia, fibularis longus, tibialis anterior, patellar ligament, iliotibial tract, hypothenar muscles, thenar muscles, flexor carpi ulnaris, flexor digitorum superficialis, palmaris longus, flexor carpi radials, brachioradialis, pronator teres, brachialis, biceps brachii, triceps brachii, pectoralis major, deltoid, trapezius, sternocleidomastoid, masseter, orbicularis oculi, temporalis, epicranial aponeurosis, teres major, extensor digitorum, extensor carpi ulnaris, anconeus, abductor policis longus, plantaris, calcanel tendon, soleus, adductor magnus, gluteus maximas, gluteus medius, latissimus dorsi, intraspinatus, and combinations thereof, and the like.

Administration of disclosed compositions can comprise, for example, injection into or in the vicinity of one or more of the following nerves, for example, the axillary nerve, phrenic nerve, spinal ganglion, spinal cord, sypathetic ganglia chain, pudendal nerve, common palmar digital nerve, ulnar nerve, deep branch of the ulnar nerve, sciatic nerve, peroneal nerve, tibial nerve, saphenous nerve, interosseous nerve, superficial peroneal nerve, intermediate dorsal cutaneous nerve, medial plantar nerve, medial dorsal cutaneous nerve, deep peroneal nerve, muscular branches of tibial nerve, intrapatellar branch of saphenous nerve, common peroneal nerve, muscular branch of femoral nerve, anterior cutaneous branches of femoral nerve, muscular branches of sciatic nerve, femoral nerve, iliolinguinal, filum terminate, iliohypogastric, obturator, ulnar, radial, obturator, radial, subcostal, intercostal, dorsal branches of the intercostal, medial cutaneous branches of the intercostal, musculaneous, deltoid, vagus, brachial plexus, supraclavicular, facial, auriculotemporal, combinations thereof, and the like.

Smooth muscles suitable for administration of disclosed compositions can comprise any of walls of blood vessels, walls of stomach, ureters, intestines, in the aorta (tunica media layer), iris of the eye, prostate, gastrointestinal tract, respiratory tract, small arteries, arterioles, reproductive tracts (both genders), veins, glomeruli of the kidneys (called mesangial cells), bladder, uterus, arrector pili of the skin, ciliary muscle, sphincter, trachea, bile ducts, and the like.

The frequency and the amount of injection under the disclosed methods can be determined based on the nature and location of the particular area being treated. In certain cases, however, repeated or supplemental injection may be desired to achieve optimal results. The frequency and the amount of the injection for each particular case can be determined by the person of ordinary skill in the art.

In embodiments, administration of the fast acting neurotoxin is performed upon a neurotoxin naïve patient prior to a surgical procedure, for example a cosmetic procedure. In embodiments, the administration is performed, for example, within 36 hours before the procedure, within 24 hours before the procedure, within 22 hours before the procedure, within 20 hours before the procedure, within 18 hours before the procedure, within 16 hours before the procedure, within 14 hours before the procedure, within 12 hours before the procedure, within 11 hours before the procedure, within 10 hours before the procedure, within 9 hours before the procedure, within 8 hours before the procedure, within 7 hours before the procedure, within 6 hours before the procedure, within 5 hours before the procedure, within 4 hours before the procedure, within 3 hours before the procedure, within 2 hours before the procedure, within 60 minutes before the procedure, within 50 minutes before the procedure, within 40 minutes before the procedure, within 30 minutes before the procedure, within 20 minutes before the procedure, within 10 minutes before the procedure, within 5 minutes before the procedure, within 2 minutes before the procedure, or the like.

In embodiments, administration of the fast acting neurotoxin is performed upon a neurotoxin naïve patient prior to a surgical procedure, for example a cosmetic procedure. In embodiments, the administration is performed, for example, not less than 36 hours before the procedure, not less than 24 hours before the procedure, not less than 22 hours before the procedure, not less than 20 hours before the procedure, not less than 18 hours before the procedure, not less than 16 hours before the procedure, not less than 14 hours before the procedure, not less than 12 hours before the procedure, not less than 11 hours before the procedure, not less than 10 hours before the procedure, not less than 9 hours before the procedure, not less than 8 hours before the procedure, not less than 7 hours before the procedure, not less than 6 hours before the procedure, not less than 5 hours before the procedure, not less than 4 hours before the procedure, not less than 3 hours before the procedure, not less than 2 hours before the procedure, not less than 60 minutes before the procedure, not less than 50 minutes before the procedure, not less than 40 minutes before the procedure, not less than 30 minutes before the procedure, not less than 20 minutes before the procedure, not less than 10 minutes before the procedure, not less than 5 minutes before the procedure, not less than 2 minutes before the procedure, or the like.

In embodiments, administration of the fast acting neurotoxin is performed concurrently with a surgical procedure, for example a cosmetic procedure.

In embodiments, administration of the fast acting neurotoxin is performed upon a patient, for example a neurotoxin naïve patient, after a surgical procedure, for example a cosmetic procedure. For example, administration can be performed, within 1 minute after the procedure, within 2 minutes after the procedure, within 3 minutes after the procedure, within 4 minutes after the procedure, within 5 minutes after the procedure, within 6 minutes after the procedure, within 7 minutes after the procedure, within 8 minutes after the procedure, within 9 minutes after the procedure, within 10 minutes after the procedure, within 20 minutes after the procedure, within 30 minutes after the procedure, within 40 minutes after the procedure, within 50 minutes after the procedure, within 60 minutes after the procedure, within 90 minutes after the procedure, within 2 hours after the procedure, within 3 hours after the procedure, within 4 hours after the procedure, within 5 hours after the procedure, within 6 hours after the procedure, within 7 hours after the procedure, within 8 hours after the procedure, within 9 hours after the procedure, within 10 hours after the procedure, within 11 hours after the procedure, within 12 hours after the procedure, or the like.

Embodiments comprise administration of a fast-acting neurotoxin following an injury. For example, in embodiments, the fast-acting neurotoxin can be administered within 5 minutes of an injury occurring, within 10 minutes an injury, within 15 minutes of an injury, within 20 minutes of an injury, within 25 minutes of an injury, within 30 minutes of an injury, within 35 minutes of an injury, within 40 minutes of an injury, within 45 minutes of an injury, within 50 minutes of an injury, within 55 minutes of an injury, within 60 minutes of an injury, within 65 minutes of an injury, within 70 minutes of an injury, within 75 minutes of an injury, within 80 minutes of an injury, within 85 minutes of an injury, within 90 minutes of an injury, within 95 minutes of an injury, within 100 minutes of an injury, within 110 minutes of an injury, within 2 hours of an injury, within 2 hours of an injury, within 3 hours of an injury, within 4 hours of an injury, within 5 hours of an injury, within 6 hours of an injury, within 7 hours of an injury, within 8 hours of an injury, within 9 hours of an injury, within 10 hours of an injury, within 11 hours of an injury, within 12 hours of an injury, within 13 hours of an injury, within 14 hours of an injury, within 15 hours of an injury, within 16 hours of an injury, within 17 hours of an injury, within 18 hours of an injury, within 19 hours of an injury, within 20 hours of an injury, within 21 hours of an injury, within 22 hours of an injury, within 23 hours of an injury, within 1 day of an injury, within 2 days of an injury, within 3 days of an injury, within 4 days of an injury, within 5 days of an injury, within 6 days of an injury, within 7 days of an injury, within 8 days of an injury, within 9 days of an injury, within 10 days of an injury, or the like.

Methods disclosed herein can comprise supplemental administration of a fast-acting neurotoxin, for example a BoNT/E, to a patient after an initial administration. Embodiments comprising supplemental administration can further comprise doctor or patient evaluation of the results of a prior neurotoxin administration. Such evaluation can comprise the use of, for example, photographs, scanning, or the like.

In embodiments, evaluation of the results of the initial neurotoxin administration can be performed within, for example, 6 hours of the initial administration, 8 hours of the initial administration, 10 hours of the initial administration, 12 hours of the initial administration, 14 hours of the initial administration, 16 hours of the initial administration, 18 hours of the initial administration, 24 hours of the initial administration, 30 hours of the initial administration, 36 hours of the initial administration, 42 hours of the initial administration, 48 hours of the initial administration, 54 hours of the initial administration, 60 hours of the initial administration, 66 hours of the initial administration, 72 hours of the initial administration, 78 hours of the initial administration, 84 hours of the initial administration, 90 hours of the initial administration, 96 hours of the initial administration, 102 hours of the initial administration, 108 hours of the initial administration, 114 hours of the initial administration, 120 hours of the initial administration, 1 week of the initial administration, 2 weeks of the initial administration, 3 weeks of the initial administration, 4 weeks of the initial administration, 5 weeks of the initial administration, 6 weeks of the initial administration, 7 weeks of the initial administration, 8 weeks of the initial administration, 9 weeks of the initial administration, 10 weeks of the initial administration, 11 weeks of the initial administration, 12 weeks of the initial administration, or the like.

In embodiments comprising a supplemental administration, administration of the supplemental dose can be performed within, for example, 6 hours of the evaluation, 8 hours of the evaluation, 10 hours of the evaluation, 12 hours of the evaluation, 14 hours of the evaluation, 16 hours of the evaluation, 18 hours of the evaluation, 24 hours of the evaluation, 30 hours of the evaluation, 36 hours of the evaluation, 42 hours of the evaluation, 48 hours of the evaluation, 54 hours of the evaluation, 60 hours of the evaluation, 66 hours of the evaluation, 72 hours of the evaluation, 78 hours of the evaluation, 84 hours of the evaluation, 90 hours of the evaluation, 96 hours of the evaluation, 102 hours of the evaluation, 108 hours of the evaluation, 114 hours of the evaluation, 120 hours of the evaluation, 1 week of the evaluation, 2 weeks of the evaluation, 3 weeks of the evaluation, 4 weeks of the evaluation, 5 weeks of the evaluation, 6 weeks of the evaluation, 7 weeks of the evaluation, 8 weeks of the evaluation, 9 weeks of the evaluation, 10 weeks of the evaluation, 11 weeks of the evaluation, 12 weeks of the evaluation, or the like.

In embodiments, the supplemental administration can be performed, for example, within, for example, 6 hours of the initial administration, 8 hours of the initial administration, 10 hours of the initial administration, 12 hours of the initial administration, 14 hours of the initial administration, 16 hours of the initial administration, 18 hours of the initial administration, 24 hours of the initial administration, 30 hours of the initial administration, 36 hours of the initial administration, 42 hours of the initial administration, 48 hours of the initial administration, 54 hours of the initial administration, 60 hours of the initial administration, 66 hours of the initial administration, 72 hours of the initial administration, 78 hours of the initial administration, 84 hours of the initial administration, 90 hours of the initial administration, 96 hours of the initial administration, 102 hours of the initial administration, 108 hours of the initial administration, 114 hours of the initial administration, 120 hours of the initial administration, 1 week of the initial administration, 2 weeks of the initial administration, 3 weeks of the initial administration, 4 weeks of the initial administration, 5 weeks of the initial administration, 6 weeks of the initial administration, 7 weeks of the initial administration, 8 weeks of the initial administration, 9 weeks of the initial administration, 10 weeks of the initial administration, 11 weeks of the initial administration, 12 weeks of the initial administration, or the like.

Methods disclosed herein can provide rapid-onset effects (for example, using a fast-acting neurotoxin, for example a BoNT/E). For example, disclosed embodiments can provide visible cosmetic effect within, for example, 30 minutes after administration, 45 minutes after administration, 60 minutes after administration, 75 minutes after administration, 90 minutes after administration, 2 hours after administration, 3 hours after administration, 4 hours after administration, 5 hours after administration, 6 hours after administration, 7 hours after administration, 8 hours after administration, 9 hours after administration, 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, 30 hours after administration, 36 hours after administration, 42 hours after administration, 48 hours after administration, 3 days after administration, 4 days after administration, 5 days after administration, 6 days after administration, 7 days after administration, 8 days after administration, 9 days after administration, 10 days after administration, 11 days after administration, 12 days after administration, or the like.

Methods disclosed herein can provide effects of a shorter direction (for example, using a fast-recovery neurotoxin, for example a BoNT/E). For example, disclosed embodiments can provide visible cosmetic effects that subside within, for example, 3 days after administration, 4 days after administration, 5 days after administration, 6 days after administration, 7 days after administration, 8 days after administration, 9 days after administration, 10 days after administration, 11 days after administration, 12 days after administration, 13 days after administration, 14 days after administration, 15 days after administration, 16 days after administration, 17 days after administration, 18 days after administration, 19 days after administration, 20 days after administration, 21 days after administration, 22 days after administration, 23 days after administration, 24 days after administration, 25 days after administration, 26 days after administration, 27 days after administration, 28 days after administration, 29 days after administration, 30 days after administration, 45 days after administration, 60 days after administration, 75 days after administration, 90 days after administration, 105 days after administration, or the like.

Side-effects can be associated with botulinum injections. Disclosed embodiments can provide neurotoxin, for example a BoNT/E, treatments that result in fewer side effects, or side effects of a shorted duration, than conventional neurotoxin treatments.

For example, disclosed embodiments can result in fewer (or shorter duration) instances of double vision or blurred vision, eyelid paralysis (subject cannot lift eyelid all the way open), loss of facial muscle movement, hoarseness, loss of bladder control, shortness of breath, difficulty in swallowing, difficulty speaking, death, and the like.

Disclosed methods can be particularly suitable for treatment of cosmetic irregularities, which are usually results of aging, environmental exposure, weight loss, child bearing, injury, surgery, or combinations thereof. Aging and environmental exposure often cause wrinkles on various positions of the skin. Weight loss and child bearing, on the other hand, often cause stretch marks on various positions of the skin, especially on stomach, areas of the lower body, and legs. Injury and surgery often result in scars in areas of injury and operation. Specific contour deficiencies suitable for treatment by the disclosed methods include, but are not limited to, frown lines, worry lines, wrinkles, crow's feet, marionette lines, stretch marks, wounds, accidents, bites, surgery, or the like. Particularly suitable for treatment according to the present invention are contour deficiencies of such areas as eyes, cheeks, nose, lips, forehead, and neck.

Further, disclosed embodiments can provide patients with cosmetic results of a more-certain duration. For example, with a longer acting neurotoxin, a 20% variance in duration of effects can result in a month's difference in effective duration. With the disclosed fast-recovery neurotoxins, this 20% variance produces a much less drastic difference in effective duration.

Supplemental administrations of a fast-acting neurotoxin, for example a BoNT/E, can effectively modify or augment previous cosmetic neurotoxin administrations. For example, methods disclosed herein can comprise a supplemental administration to correct an unfavorable cosmetic result from a previous administration, or to increase the cosmetic effects of a previous administration, or to accelerate the onset of results as compared to those achieved using non fast-acting neurotoxins.

The fast-acting neurotoxin, for example a BoNT/E, can be administered in an amount of between about 10⁻³ U/kg and about 35 U/kg body weight. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 500 units of a neurotoxin, such as a botulinum type E, provides effective therapeutic or cosmetic relief. In an embodiment, from about 5 units to about 200 units of a neurotoxin, such as a botulinum type E, can be used and in another embodiment, from about 10 units to about 100 units of a neurotoxin, such as a botulinum type E, can be locally administered into a target tissue such as a muscle.

In embodiments, administration can comprise a dose of about 4 units of a neurotoxin, or about 5 units of a neurotoxin, or about 6 units of a neurotoxin, or about 7 units of a neurotoxin, or about 8 units of a neurotoxin, or about 10 units of a neurotoxin, or about 15 units of a neurotoxin, or about 20 units of a neurotoxin, or about 30 units of a neurotoxin, or about 40 units of a neurotoxin, or about 50 units of a neurotoxin, or about 60 units of a neurotoxin, or about 70 units of a neurotoxin, or about 80 units of a neurotoxin, or about 90 units of a neurotoxin, or about 100 units of a neurotoxin, or about 110 units of a neurotoxin, or about 120 units of a neurotoxin, or about 130 units of a neurotoxin, or about 140 units of a neurotoxin, or about 150 units of a neurotoxin, or about 160 units of a neurotoxin, or about 170 units of a neurotoxin, or about 180 units of a neurotoxin, or about 190 units of a neurotoxin, or about 200 units of a neurotoxin, or about 210 units of a neurotoxin, or about 220 units of a neurotoxin, or about 230 units of a neurotoxin, or about 240 units of a neurotoxin, or about 250 units of a neurotoxin, or about 260 units of a neurotoxin, or about 270 units of a neurotoxin, or about 280 units of a neurotoxin, or about 290 units of a neurotoxin, or about 290 units of a neurotoxin, or about 300 units of a neurotoxin, or about 310 units of a neurotoxin, or about 320 units of a neurotoxin, or about 330 units of a neurotoxin, or about 340 units of a neurotoxin, or about 350 units of a neurotoxin, or about 360 units of a neurotoxin, or about 370 units of a neurotoxin, or about 380 units of a neurotoxin, or about 390 units of a neurotoxin, or about 400 units of a neurotoxin, or about 410 units of a neurotoxin, or about 420 units of a neurotoxin, or about 430 units of a neurotoxin, or about 440 units of a neurotoxin, or about 450 units of a neurotoxin, or about 460 units of a neurotoxin, or about 470 units of a neurotoxin, or about 480 units of a neurotoxin, or about 490 units of a neurotoxin, or about 500 units of a neurotoxin, or the like.

In embodiments, administration can comprise a dose of about 4 units of a botulinum type E neurotoxin, or about 5 units of a botulinum type E neurotoxin, or about 6 units of a botulinum type E neurotoxin, or about 7 units of a botulinum type E neurotoxin, or about 8 units of a botulinum type E neurotoxin, or about 10 units of a botulinum type E neurotoxin, or about 15 units of a botulinum type E neurotoxin, or about 20 units of a botulinum type E neurotoxin, or about 30 units of a botulinum type E neurotoxin, or about 40 units of a botulinum type E neurotoxin, or about 50 units of a botulinum type E neurotoxin, or about 60 units of a botulinum type E neurotoxin, or about 70 units of a botulinum type E neurotoxin, or about 80 units of a botulinum type E neurotoxin, or about 90 units of a botulinum type E neurotoxin, or about 100 units of a botulinum type E neurotoxin, or about 110 units of a botulinum type E neurotoxin, or about 120 units of a botulinum type E neurotoxin, or about 130 units of a botulinum type E neurotoxin, or about 140 units of a botulinum type E neurotoxin, or about 150 units of a botulinum type E neurotoxin, or about 160 units of a botulinum type E neurotoxin, or about 170 units of a botulinum type E neurotoxin, or about 180 units of a botulinum type E neurotoxin, or about 190 units of a botulinum type E neurotoxin, or about 200 units of a botulinum type E neurotoxin, or about 210 units of a botulinum type E neurotoxin, or about 220 units of a botulinum type E neurotoxin, or about 230 units of a botulinum type E neurotoxin, or about 240 units of a botulinum type E neurotoxin, or about 250 units of a botulinum type E neurotoxin, or about 260 units of a botulinum type E neurotoxin, or about 270 units of a botulinum type E neurotoxin, or about 280 units of a botulinum type E neurotoxin, or about 290 units of a botulinum type E neurotoxin, or about 290 units of a botulinum type E neurotoxin, or about 300 units of a botulinum type E neurotoxin, or about 310 units of a botulinum type E neurotoxin, or about 320 units of a botulinum type E neurotoxin, or about 330 units of a botulinum type E neurotoxin, or about 340 units of a botulinum type E neurotoxin, or about 350 units of a neurotoxin, or about 360 units of a botulinum type E neurotoxin, or about 370 units of a botulinum type E neurotoxin, or about 380 units of a botulinum type E neurotoxin, or about 390 units of a botulinum type E neurotoxin, or about 400 units of a botulinum type E neurotoxin, or about 410 units of a botulinum type E neurotoxin, or about 420 units of a botulinum type E neurotoxin, or about 430 units of a botulinum type E neurotoxin, or about 440 units of a botulinum type E neurotoxin, or about 450 units of a botulinum type E neurotoxin, or about 460 units of a botulinum type E neurotoxin, or about 470 units of a botulinum type E neurotoxin, or about 480 units of a botulinum type E neurotoxin, or about 490 units of a botulinum type E neurotoxin, or about 500 units of a botulinum type E neurotoxin, or the like.

Disclosed herein are methods for expressing neurotoxin dosages and conveying neurotoxin dosage amounts. In embodiments, the dosage amount is expressed in protein amount, for example nanograms (ng). In embodiments, the neurotoxin can comprise a botulinum toxin. In embodiments, the dosage amount is expressed in picograms (pg), for example between 4 and 40 pg/kg of said neurotoxin, or between 7 and 35 pg/kg, between 10 and 30 pg/kg, between 14 and 25 pg/kg, between 14 and 20 pg/kg, between 12 and 20 pg/kg, between 10 and 30 pg/kg, or the like.

Methods disclosed herein can comprise administration of a neurotoxin, for example a fast-acting neurotoxin, to a patient, wherein the dosage of the neurotoxin is expressed in protein amount, for example protein amount per administration. In an embodiment the fast-acting neurotoxin is a botulinum toxin, for example BoNT/E.

In embodiments, the dose of the neurotoxin, for example BoNT/E, is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like into a target tissue such as a muscle.

In embodiments, administration can comprise a total dose of between 5 and 7 ng, between 7 and 9 ng, between 9 and 11 ng, between 11 and 13 ng, between 13 and 15 ng, between 15 and 17 ng, between 17 and 19 ng, or the like.

In embodiments, administration can comprise a total dose of not more than 5 ng, not more than 6 ng, not more than 7 ng, not more than 8 ng, not more than 9 ng, not more than 10 ng, not more than 11 ng, not more than 12 ng, not more than 13 ng, not more than 14 ng, not more than 15 ng, not more than 16 ng, not more than 17 ng, not more than 18 ng, not more than 19 ng, not more than 20 ng, not more than 22 ng, not more than 24 ng, not more than 26 ng, not more than 28 ng, not more than 30 ng, not more than 32 ng, not more than 34 ng, not more than 36 ng, not more than 38 ng, not more than 40 ng, not more than 42 ng, not more than 44 ng, not more than 46 ng, not more than 48 ng, not more than 50 ng, not more than 52 ng, not more than 54 ng, not more than 56 ng, not more than 58 ng, not more than 60 ng, not more than 62 ng, not more than 64 ng, not more than 66 ng, not more than 68 ng, not more than 70 ng, not more than 72 ng, not more than 74 ng, not more than 76 ng, not more than 78 ng, not more than 80 ng, not more than 82 ng, not more than 84 ng, not more than 86 ng, not more than 88 ng, not more than 90 ng, not more than 92 ng, not more than 94 ng, not more than 96 ng, not more than 98 ng, not more than 100 ng, or the like.

In embodiments, administration can comprise a total dose of not less than 5 ng, not less than 6 ng, not less than 7 ng, not less than 8 ng, not less than 9 ng, not less than 10 ng, not less than 11 ng, not less than 12 ng, not less than 13 ng, not less than 14 ng, not less than 15 ng, not less than 16 ng, not less than 17 ng, not less than 18 ng, not less than 19 ng, not less than 20 ng, not less than 22 ng, not less than 24 ng, not less than 26 ng, not less than 28 ng, not less than 30 ng, not less than 32 ng, not less than 34 ng, not less than 36 ng, not less than 38 ng, not less than 40 ng, not less than 42 ng, not less than 44 ng, not less than 46 ng, not less than 48 ng, not less than 50 ng, not less than 52 ng, not less than 54 ng, not less than 56 ng, not less than 58 ng, not less than 60 ng, not less than 62 ng, not less than 64 ng, not less than 66 ng, not less than 68 ng, not less than 70 ng, not less than 72 ng, not less than 74 ng, not less than 76 ng, not less than 78 ng, not less than 80 ng, not less than 82 ng, not less than 84 ng, not less than 86 ng, not less than 88 ng, not less than 90 ng, not less than 92 ng, not less than 94 ng, not less than 96 ng, not less than 98 ng, not less than 100 ng, or the like.

In embodiments, administration can comprise a total dose of about 0.1 ng of a botulinum type E neurotoxin, 0.2 ng of a neurotoxin, 0.3 ng of a neurotoxin, 0.4 ng of a neurotoxin, 0.5 ng of a neurotoxin, 0.6 ng of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 ng of a neurotoxin, 3.4 ng of a neurotoxin, 3.5 ng of a neurotoxin, 3.6 ng of a neurotoxin, 3.7 ng of a neurotoxin, 3.8 ng of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, 11 ng of a neurotoxin, 12 ng of a neurotoxin, 13 ng of a neurotoxin, 14 ng of a neurotoxin, 15 ng of a neurotoxin, 16 ng of a neurotoxin, 17 ng of a neurotoxin, 18 ng of a neurotoxin, 19 ng of a neurotoxin, 20 ng of a neurotoxin, 20 ng of a neurotoxin, 22 ng of a neurotoxin, 24 ng of a neurotoxin, 26 ng of a neurotoxin, 28 ng of a neurotoxin, 30 ng of a neurotoxin, 32 ng of a neurotoxin, 34 ng of a neurotoxin, 36 ng of a neurotoxin, 38 ng of a neurotoxin, 40 ng of a neurotoxin, 42 ng of a neurotoxin, 44 ng of a neurotoxin, 46 ng of a neurotoxin, 48 ng of a neurotoxin, 50 ng of a neurotoxin, 52 ng of a neurotoxin, 54 ng of a neurotoxin, 56 ng of a neurotoxin, 58 ng of a neurotoxin, 60 ng of a neurotoxin, 62 ng of a neurotoxin, 64 ng of a neurotoxin, 66 ng of a neurotoxin, 68 ng of a neurotoxin, 70 ng of a neurotoxin, 72 ng of a neurotoxin, 74 ng of a neurotoxin, 76 ng of a neurotoxin, 78 ng of a neurotoxin, 80 ng of a neurotoxin, 82 ng of a neurotoxin, 84 ng of a neurotoxin, 86 ng of a neurotoxin, 88 ng of a neurotoxin, 90 ng of a neurotoxin, 92 ng of a neurotoxin, 94 ng of a neurotoxin, 96 ng of a neurotoxin, 98 ng of a neurotoxin, 100 ng of a neurotoxin, or the like.

In embodiments, administration can comprise a dose per injection of for example, about 0.1 ng of a botulinum type E neurotoxin, 0.2 ng of a botulinum type E neurotoxin, 0.3 ng of a botulinum type E neurotoxin, 0.4 ng of a botulinum type E neurotoxin, 0.5 ng of a botulinum type E neurotoxin, 0.6 ng of a botulinum type E neurotoxin, 0.7 ng of a botulinum type E neurotoxin, 0.8 ng of a botulinum type E neurotoxin, 0.9 ng of a botulinum type E neurotoxin, 1.0 ng of a botulinum type E neurotoxin, 1.1 ng of a botulinum type E neurotoxin, 1.2 ng of a botulinum type E neurotoxin, 1.3 ng of a botulinum type E neurotoxin, 1.4 ng of a botulinum type E neurotoxin, 1.5 ng of a botulinum type E neurotoxin, 1.6 ng of a botulinum type E neurotoxin, 1.7 ng of a botulinum type E neurotoxin, 1.8 ng of a botulinum type E neurotoxin, 1.9 ng of a botulinum type E neurotoxin, 2.0 ng of a botulinum type E neurotoxin, 2.1 ng of a botulinum type E neurotoxin, 2.2 ng of a botulinum type E neurotoxin, 2.3 ng of a botulinum type E neurotoxin, 2.4 ng of a botulinum type E neurotoxin, 2.5 ng of a botulinum type E neurotoxin, 2.6 ng of a botulinum type E neurotoxin, 2.7 ng of a botulinum type E neurotoxin, 2.8 ng of a botulinum type E neurotoxin, 2.9 ng of a botulinum type E neurotoxin, 3.0 ng of a botulinum type E neurotoxin, 3.1 ng of a botulinum type E neurotoxin, 3.2 ng of a botulinum type E neurotoxin, 3.3 ng of a botulinum type E neurotoxin, 3.4 ng of a botulinum type E neurotoxin, 3.5 ng of a botulinum type E neurotoxin, 3.6 ng of a botulinum type E neurotoxin, 3.7 ng of a botulinum type E neurotoxin, 3.8 ng of a botulinum type E neurotoxin, 3.9 ng of a botulinum type E neurotoxin, 4.0 ng of a botulinum type E neurotoxin, 4.1 ng of a botulinum type E neurotoxin, 4.2 ng of a botulinum type E neurotoxin, 4.3 ng of a botulinum type E neurotoxin, 4.4 ng of a botulinum type E neurotoxin, 4.5 ng of a botulinum type E neurotoxin, 5 ng of a botulinum type E neurotoxin, 6 ng of a botulinum type E neurotoxin, 7 ng of a botulinum type E neurotoxin, 8 ng of a botulinum type E neurotoxin, 9 ng of a botulinum type E neurotoxin, 10 ng of a botulinum type E neurotoxin, or the like.

In embodiments, administration can comprise a total dose of not more than 5 pg, not more than 6 pg, not more than 7 pg, not more than 8 pg, not more than 9 pg, not more than 10 pg, not more than 11 pg, not more than 12 pg, not more than 13 pg, not more than 14 pg, not more than 15 pg, not more than 16 pg, not more than 17 pg, not more than 18 pg, not more than 19 pg, not more than 20 pg, or the like.

In embodiments, administration can comprise a total dose of not less than 5 pg, not less than 6 pg, not less than 7 pg, not less than 8 pg, not less than 9 pg, not less than 10 pg, not less than 11 pg, not less than 12 pg, not less than 13 pg, not less than 14 pg, not less than 15 pg, not less than 16 pg, not less than 17 pg, not less than 18 pg, not less than 19 pg, not less than 20 pg, or the like.

Ultimately, however, both the quantity of toxin administered and the frequency of its administration will be at the discretion of the physician responsible for the treatment and will be commensurate with questions of safety and the effects produced by the toxin.

In embodiments, the dose of the opioid can be, for example, between 0.1 and 100 mg, between 1 and 100 mg, between 4 and 95 mg, between 6 and 90 mg, between 8 and 85 mg, between 10 and 80 mg, between 20 and 60 mg, and the like.

In embodiments, the dose of the opioid can be, for example, at least 1 mg, at least 2 mg, at least 4 mg, at least 6 mg, at least 8 mg, at least 10 mg, at least 12 mg, at least 14 mg, at least 16 mg, at least 18 mg, at least 20 mg, at least 22 mg, at least 24 mg, at least 26 mg, at least 28 mg, at least 30 mg, at least 32 mg, at least 34 mg, at least 36 mg, at least 38 mg, at least 40 mg, at least 42 mg, at least 44 mg, at least 46 mg, at least 48 mg, at least 50 mg, at least 55 mg, at least 60 mg, at least 65 mg, at least 70 mg, at least 75 mg, at least 80 mg, at least 85 mg, at least 95 mg, at least 100 mg, or the like.

In embodiments, the dose of the opioid can be, for example, not more than 1 mg, not more than 2 mg, not more than 4 mg, not more than 6 mg, not more than 8 mg, not more than 10 mg, not more than 12 mg, not more than 14 mg, not more than 16 mg, not more than 18 mg, not more than 20 mg, not more than 22 mg, not more than 24 mg, not more than 26 mg, not more than 28 mg, not more than 30 mg, not more than 32 mg, not more than 34 mg, not more than 36 mg, not more than 38 mg, not more than 40 mg, not more than 42 mg, not more than 44 mg, not more than 46 mg, not more than 48 mg, not more than 50 mg, not more than 55 mg, not more than 60 mg, not more than 65 mg, not more than 70 mg, not more than 75 mg, not more than 80 mg, not more than 85 mg, not more than 95 mg, not more than 100 mg, or the like.

In embodiments comprising anesthetics, methods can comprise administration of a pre-anesthetic medication. For example, prior to administration of the anesthetic, a patient can be administered an antacid, an H₂ blocker, and anticholinergic, an antiemetic, an antihistamine, combinations thereof, or the like.

In embodiments, the anesthetic can be administered topically, locally, via inhalation, intravenously, or the like.

In embodiments the dose of the anesthetic can be, for example, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.2 mg/kg, 1.4 mg/kg, 1.6 mg/kg, 1.8 mg/kg, 2 mg/kg, 4 mg/kg, 6 mg/kg, 8 mg/kg, 10 mg/kg, or the like.

In embodiments the dose of the anesthetic can be, for example, 0.005 mg/kg per day, 0.01 mg/kg per day, 0.02 mg/kg per day, 0.03 mg/kg per day, 0.04 mg/kg per day, 0.05 mg/kg per day, 0.06 mg/kg per day, 0.07 mg/kg per day, 0.08 mg/kg per day, 0.09 mg/kg per day, 0.1 mg/kg per day, 0.2 mg/kg per day, 0.3 mg/kg per day, 0.4 mg/kg per day, 0.5 mg/kg per day, 0.6 mg/kg per day, 0.7 mg/kg per day, 0.8 mg/kg per day, 0.9 mg/kg per day, 1 mg/kg per day, 1.2 mg/kg per day, 1.4 mg/kg per day, 1.6 mg/kg per day, 1.8 mg/kg per day, 2 mg/kg per day, 4 mg/kg per day, 6 mg/kg per day, 8 mg/kg per day, 10 mg/kg per day, or the like.

In embodiments the dose of the anesthetic can be, for example, 0.005 mg/kg/hr, 0.01 mg/kg/hr, 0.02 mg/kg/hr, 0.03 mg/kg/hr, 0.04 mg/kg/hr, 0.05 mg/kg/hr, 0.06 mg/kg/hr, 0.07 mg/kg/hr, 0.08 mg/kg/hr, 0.09 mg/kg/hr, 0.1 mg/kg/hr, 0.2 mg/kg/hr, 0.3 mg/kg/hr, 0.4 mg/kg/hr, 0.5 mg/kg/hr, 0.6 mg/kg/hr, 0.7 mg/kg/hr, 0.8 mg/kg/hr, 0.9 mg/kg/hr, 1 mg/kg/hr, 1.2 mg/kg/hr, 1.4 mg/kg/hr, 1.6 mg/kg/hr, 1.8 mg/kg/hr, 2 mg/kg/hr, 4 mg/kg/hr, 6 mg/kg/hr, 8 mg/kg/hr, 10 mg/kg/hr, or the like.

A kit for practicing disclosed embodiments is also encompassed by the present disclosure. The kit can comprise a 30 gauge or smaller needle and a corresponding syringe. The kit also comprises a Clostridial neurotoxin composition, such as a botulinum type E toxin composition. The neurotoxin composition may be provided in the syringe. The composition is injectable through the needle. The kits are designed in various forms based the sizes of the syringe and the needles and the volume of the injectable composition contained therein, which in turn are based on the specific cosmetic deficiencies the kits are designed to treat.

Methods disclosed herein can comprise administration of a slower-acting, slower-recovery neurotoxin following an evaluation time period that follows administration of the fast-acting neurotoxin to a patient. During the evaluation period, the patient can decide whether to pursue further neurotoxin treatments, for example treatment with BoNT/E, or treatment with BoNT/A. In embodiments, the evaluation time period can be, for example, 6 hours following the initial administration of the fast-acting, fast-recovery neurotoxin, 8 hours following the initial administration, 10 hours following the initial administration, 12 hours following the initial administration, 14 hours following the initial administration, 16 hours following the initial administration, 18 hours following the initial administration, 24 hours following the initial administration, 30 hours following the initial administration, 36 hours following the initial administration, 42 hours following the initial administration, 48 hours following the initial administration, 54 hours following the initial administration, 60 hours following the initial administration, 66 hours following the initial administration, 72 hours following the initial administration, 78 hours following the initial administration, 84 hours following the initial administration, 90 hours following the initial administration, 96 hours following the initial administration, 102 hours following the initial administration, 108 hours following the initial administration, 114 hours following the initial administration, 120 hours following the initial administration, 1 week following the initial administration, 2 weeks following the initial administration, 3 weeks following the initial administration, 4 weeks following the initial administration, 5 weeks following the initial administration, 6 weeks following the initial administration, 7 weeks following the initial administration, 8 weeks following the initial administration, 9 weeks following the initial administration, 10 weeks following the initial administration, 11 weeks following the initial administration, 12 weeks following the initial administration, or the like.

In embodiments, the evaluation time period can be, for example, 2 weeks following the initial administration of the fast-acting, fast-recovery neurotoxin, 3 weeks following the initial administration, 4 weeks following the initial administration, 5 weeks following the initial administration, 6 weeks following the initial administration, 7 weeks following the initial administration, 8 weeks following the initial administration, 9 weeks following the initial administration, 10 weeks following the initial administration, 11 weeks following the initial administration, 12 weeks following the initial administration, 13 weeks following the initial administration, 14 weeks following the initial administration, 15 weeks following the initial administration, 16 weeks following the initial administration, or the like.

### EXAMPLES

The following non-limiting examples are provided for illustrative purposes only in order to facilitate a more complete understanding of representative embodiments. This example should not be construed to limit any of the embodiments described in the present specification.

### Example 1—Thermo-Reversible Gel Formulation

In a prophetic example, a poly(ethylene glycol)-poly-(serinol hexamethylene urethane) (ESHU) thermal gel is prepared as described previously (Park D, Wu W, Wang Y. A functionalizable reverse thermal gel based on a polyurethane/PEG block copolymer. Biomaterials. 2011; 32:777-786). To prepare botulinum-loaded ESHU, the desired amount of polymer is measured out and sterilized via ethylene oxide (12-hour cycle, 208° C., >35% relative humidity). The sterilized ESHU polymer then is dissolved in a solutions of BoNT/E and BoNT/A, in the dark, at a polymer concentration of 15% wt/vol. The thermal gel will cause the BoNT to remain localized and extend the duration of action.

The thermo-gel formulation can be used to treat a patient for an indication where an onset of within one day is desired and duration of action of longer than three months is also desired. Examples of such indications include blepharospasm, strabismus, dystonia, hyperhidrosis, migraine, over-active bladder, upper and lower limb spasticity, essential tremors, glabellar lines, canthal lines, downturned mouth, skeletal muscle pain, and juvenile cerebral palsy.

### Example 2—Lyophilized BoNT/A and BoNT/E

In a prophetic example, the 100 U BoNT/A is lyophilized in a vial with trehalose and to form a solid that can be reconstituted. 50 U BoNT/E is separately lyophilized with trehalose, magnesium stearate, mannitol and food coloring to create a rapidly dissolving film. The BoNT/E film is placed on top of the BoNT/A solid in the vial. Upon reconstitution, both actives mix together.

The BoNT/E film can be removed for testing activity of BoNT/A and BoNT/E separately. The food coloring allows easy detection visual detection of any breakage of the BoNT/E film during removal.

### Example 3—Lyophilized BoNT/A and BoNT/E

In a prophetic example, the following components are lyophilized together to form a solid that can be reconstituted to produce a 500 U solution BoNT/A and 500 U BoNT/E: 500 U BoNT/A(+complex); 125 pg HSA; 2.5 mg lactose; 500 U solution: 500 U BoNT/E; 125 pg HSA; 2.5 mg mannitol.

### Example 4—Surgical Treatment

In a prophetic example, a 35 year-old woman undergoes breast augmentation surgery. During surgery, the muscles in the vicinity of the tissue that will be affected (e.g. the pectoralis major) are injected with 100 U of BoNT/E in combination with 200 U of BoNT/A. The practitioner reconstitutes a formulation as disclosed in Example 2.

By the time the surgical anesthetic wears off, the neurotoxins will have almost completely eliminated activity in the injected muscles. This reduces the pain experienced by the patient, and thus lessens the necessity for pain medication. Muscle activity is in the treated area is greatly reduced for 3 months.

### Example 5—Cosmetic Treatment

In a prophetic example, a 45 year-old man desires to have fewer facial wrinkles. A practitioner reconstitutes a BoNT/A and BoNT/E combination formulation as disclosed in Example 1, and administers the formulation to the relevant facial muscles as recommended for glabellar and cathal lines. The next day the patient notices a decrease in facial lines. The decrease in

### Example 6—Use of Botulinum Toxin Type E to Treat Glabellar Lines (GL)

This first-in-human, randomized, double-blinded, placebo-controlled, ascending dose cohort study enrolled 42 subjects who received EB-001 (a botulinum type E composition disclosed herein) (N=35) or placebo (N=7). The efficacy primary outcome was the proportion of subjects with a 2-grade investigator-rated (IR-2) improvement in GL severity at maximum frown. Safety evaluations included adverse events (AEs), laboratory tests, and physical examinations. An IR-2 response was observed starting in the third cohort (EB-001), with increased rates observed at higher doses. Onset of clinical effect was within 24 hours, with a duration ranging between 14 and 30 days for the highest doses. AE incidence was low, with the most common being mild to moderate headache. There were no serious AEs or ptosis, and no clinically significant changes in other safety assessments.

In this clinical study in GL, EB-001 showed favorable safety and tolerability, and dose dependent efficacy with an 80% response rate at the highest dose. EB-001 maximum clinical effect was seen within 24 hours and lasted between 14 and 30 days. This differentiated EB-001 profile supports its development for aesthetic and therapeutic applications where fast onset and short duration of effect are desirable.

Botulinum neurotoxins, which inhibit the pre-synaptic release of acetylcholine, are among the most potent molecules in nature. When injected into muscles, Botulinum neurotoxins inhibit neuromuscular transmission and produce dose-dependent local muscle relaxation. Purified Botulinum neurotoxins, including serotypes A and B have been developed as injectable drugs and are widely used to treat a variety of neuromuscular conditions. Botulinum neurotoxin serotype E is a novel serotype that has not been developed for clinical use to date. Botulinum toxin type E has the fastest onset and the shortest duration of action of all the Botulinum neurotoxins. Type E has similar domain structure to type A, consisting of 2 protein chains, a 100 kDa heavy chain and a 50 kDa light chain linked by a disulfide bond.2 Type E inhibits neuromuscular transmission by cleaving the same presynaptic vesicular protein (synaptosomal associated protein 25) as type A, but at a different cleavage site. Two binding sites on motor axons mediate the high affinity recognition of nerve cells by Botulinum neurotoxins. Binding is mediated first by cell surface gangliosides and then by specific protein receptors. These receptors are found on motor axon terminals at the neuromuscular junction. Botulinum toxin types A and E have both been shown to bind the specific receptor synaptic vesicle protein 2, and only these two serotypes share this receptor. This was the first clinical study to evaluate the safety and efficacy of ascending doses of Botulinum toxin type E in subjects with GL.

This study was a first-in-human evaluation of the safety and efficacy of EB-001 and focused on the treatment of moderate to severe GL. EB-001 is a proprietary purified form of Botulinum toxin type E, formulated as a liquid for injection (Bonti, Inc., Newport Beach, Calif., USA). This was a randomized, double-blinded, placebo-controlled, ascending-dose cohort study conducted at 2 expert clinical centers (Steve Yoelin, MD Medical Associates, Newport Beach, Calif., USA; Center for Dermatology Clinical Research, Fremont, Calif., USA). This study was approved by an Institutional Review Board (Aspire Institutional Review Board, Santee, Calif., USA) and was conducted in accordance with the guidelines set by the Declaration of Helsinki. Written informed consent was received from all subjects prior to their participation.

A total of 42 healthy toxin-naïve male and female subjects, ages 18 to 60 years, were enrolled in the study. Each subject's participation was to last approximately 6 weeks. The main inclusion criteria were: the presence of bilaterally symmetrical GL of moderate to severe rating at maximum frown, sufficient visual acuity without the use of eyeglasses (contact lens use acceptable) to accurately assess their facial wrinkles, and the ability to conform with study requirements. The main criteria for exclusion were: any uncontrolled systemic disease or other medical condition, any medical condition that may have put the subject at increased risk with exposure to Botulinum neurotoxin (including diagnosed myasthenia gravis, Eaton-Lambert syndrome, amyotrophic lateral sclerosis, or any other condition that interfered with neuromuscular function), current or prior Botulinum neurotoxin treatment, known immunization or hypersensitivity to Botulinum neurotoxin, pre-specified dermatological procedures within 3 to 12 months of the study (non-ablative resurfacing, facial cosmetic procedures, topical/oral retinoid therapy, etc.), and prior periorbital surgery or treatment. Women were not enrolled if they were pregnant, lactating, or planning to become pregnant. Men with female partner(s) of childbearing potential were enrolled only if they agreed to use dual methods of contraception for 3 months following dosing.

At Screening, subject demographics, medical history, and prior and concomitant medications were recorded and an alcohol/drug screen was performed. Standardized facial photography was performed at Baseline prior to treatment, and at every follow-up visit through the end of the study, but the photographs were not used for efficacy evaluations.

Seven cohorts (6 subjects per cohort) were enrolled and received ascending doses of EB-001 or placebo in a 5:1 ratio. The maximum recommended starting dose (with a 10-fold safety factor) in this first-in-human study was developed based on the no observed adverse effect levels from a preclinical safety and toxicity study (unpublished data). From this, a base dose (Cohort 1) was calculated and determined to be sub-efficacious, and Cohorts 2 to 7 received 3, 9, 12, 16, 21, and 28 times the base dose, respectively. This represented sub-efficacious to maximum-efficacious doses of EB-001. The total dose was delivered at 5 injection sites in equal volumes (0.1 mL per site into the procerus, left and right medial corrugators, and left and right lateral corrugators) in a standardized fashion (see FIG. 1). The spacing of injections into the lateral corrugators was approximately 1 cm above the supraorbital ridge. EB-001 was supplied in a sterile solution for injection in a 5-mL vial. The placebo was supplied in identical vials without EB-001.

Each subject completed visits at Screening (Day −30 to −1), Baseline/Injection (Day 0), Days 1, 2, 7, 14, and 30 (end of study), and Day 42 (final safety follow-up).

Safety was evaluated by adverse events (AEs), laboratory testing, electrocardiograms (ECGs), physical examinations, vital signs (pulse rate, respiratory rate, and blood pressure), urine pregnancy tests (for women of childbearing potential), and focused neurologic examinations to evaluate for the potential spread of Botulinum neurotoxin. Treatment-emergent AEs (TEAEs) were defined as any AE that started or worsened in severity after exposure to study treatment. AEs and TEAEs were summarized by system organ class and preferred term using the Medical Dictionary for Regulatory Activities (MedDRA, version 19.0). Serious AEs (SAEs, or AEs that fulfilled regulatory criteria for medical seriousness), and discontinuation due to AEs were also evaluated. Severity of AEs was recorded as mild, moderate, severe, or life threatening. Before enrollment of each dosing cohort, a safety data review committee met to analyze all safety data from the previous cohort(s).

At Screening, Baseline, and Days 1, 2, 7, 14, and 30, the subject's GL were assessed at maximum frown and at rest using the Facial Wrinkle Scale (FWS). Evaluations were completed by the investigator and the subject. The FWS is a widely accepted measure used for the evaluation of facial line severity. In the present study, the 4-point scale indicating severity of GL was as follows: 0=none, 1=mild, 2=moderate, 3=severe. Subjects were considered as treatment responders if they achieved at least a 2-grade improvement (reduction) based on the investigator's FWS assessment (IR-2). The primary efficacy variable was the proportion of IR-2 responders at maximum frown at any post baseline visit through Day 30. An additional efficacy endpoint of interest was the proportion of responders achieving an investigator-assessed FWS grade of none or mild at Days 1, 2, 7, 14, or 30 (analyzed by visit).

Two analysis populations were pre-specified, a safety and an efficacy population. Subjects receiving placebo were pooled for all analyses. The safety population included all subjects who received study treatment and had at least 1 safety assessment thereafter. All TEAEs and SAEs were summarized by treatment group. All safety parameters, including laboratory testing, ECGs, physical exams, vital signs, urine pregnancy tests, and focused neurologic examinations, were reviewed and evaluated for clinical significance by the investigators. The efficacy population was the modified intent-to-treat (mITT) population, defined as all randomized subjects who received at least 1 dose of study treatment and had at least 1 post baseline efficacy assessment. Analyses of demographics and baseline characteristics were performed on the mITT population. Medical history was based on the safety population and coded using MedDRA and summarized by system organ class and preferred term. Prior and concomitant medications were based on the safety population and coded using the World Health Organization Anatomical Therapeutic Chemical classification index and summarized by drug class and treatment group. Efficacy analyses were performed using the mITT population. FWS grades were summarized by treatment and study day using frequency counts and rates of response (%). An analysis comparing the proportion of IR-2 responders in each EB-001 cohort versus placebo (pooled) was performed using Fisher's exact test with a 0.05 level of significance.

Of the 59 subjects who were screened for the study, 43 were enrolled into 1 of 7 cohorts. One subject did not receive treatment, and consequently 42 subjects were included in the mITT and safety populations (35 treated with EB-001 and 7 treated with placebo). Forty-one subjects completed the study, with 1 subject lost to follow-up. The demographic and baseline characteristics of the mITT population are displayed in Table 1. The mean (range) ages of subjects for the EB-001 (pooled) versus placebo (pooled) groups were 47.9 (22 to 60) and 50.4 (32 to 57) years, respectively. The majority of subjects were female (EB-001=91.4%; placebo=85.7%) and white (71.4% for both groups). The baseline mean (standard deviation [SD]) investigator-assessed GL at maximum frown were 2.6 (0.50) and 2.9 (0.38) for the EB-001 and placebo groups, respectively. The EB-001 and placebo groups were well balanced with no substantial between-group differences.

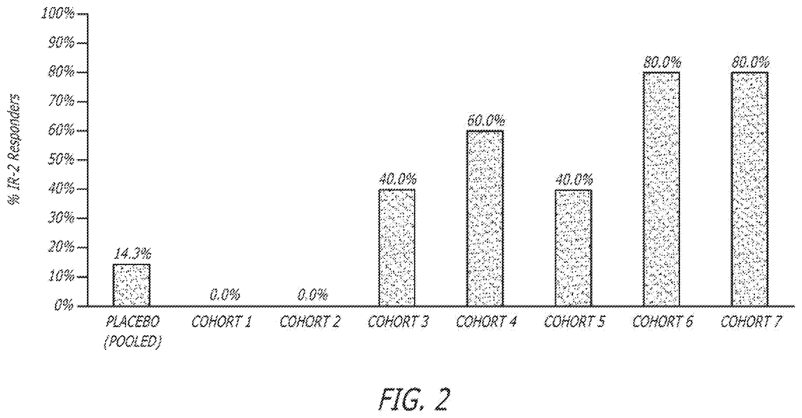

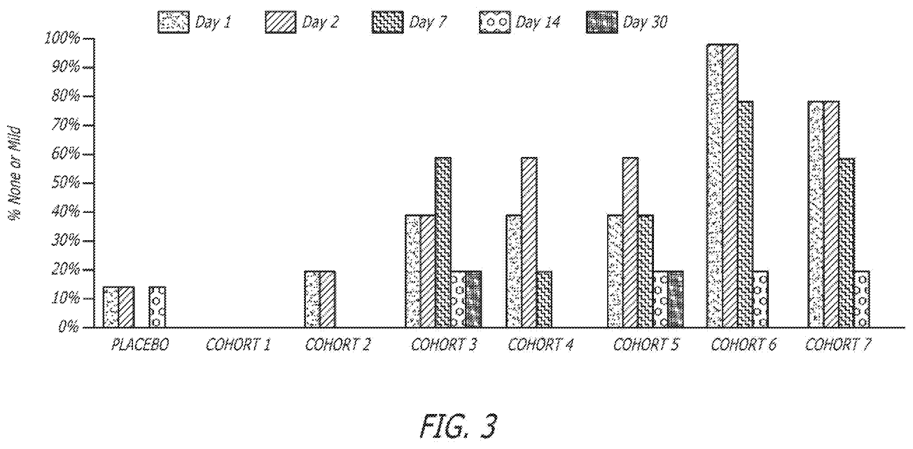

The proportions of subjects in the mITT population achieving an IR-2 response for GL severity at maximum frown at any postbaseline visit through Day 30 are presented by dose cohort in FIG. 2. In Cohort 3, 40% of subjects were IR-2 responders. This responder rate was the same or greater in all higher dose cohorts, with Cohorts 6 and 7 having 80% IR-2 responders. Cohorts 6 and 7 demonstrated significantly greater percentages of IR-2 responders versus placebo (P=0.046). FIG. 3 summarizes the proportions of subjects in each cohort with investigator-assessed FWS grades of none or mild GL at maximum frown, at any post baseline visit through Day 30. Cohorts 2 to 7 (inclusive) had greater percentages of responders versus placebo, with rates of 60% to 100% achieved for Cohorts 3 and higher. In Cohorts 3 to 7, most none or mild responses were observed at Days 1, 2, and/or 7. One responder (20%) was observed at Day 14 in Cohorts 3, 5, 6 and 7 and at Day 30 in Cohorts 3 and 5. The safety results support the safety of all evaluated doses of EB-001, administered as IM injections, in this population. No clinically significant changes from baseline in neurologic examinations, ECGs, physical examinations, or laboratory tests were observed for any subject.

Five subjects treated with EB-001 reported TEAEs, and none in placebo group. No SAEs were reported and no TEAE led to discontinuation of the study. All TEAEs were mild or moderate in severity. The events of sore throat and flu like symptoms were considered unrelated to treatment. Three subjects reported TEAEs of headache, 1 of which was considered related to treatment. There was no dose-related increase in the incidence of headaches. There were no events of ptosis or other TEAE possibly related to spread of toxin.

To our knowledge, this is the first controlled clinical trial of a Botulinum toxin type E product in any aesthetic or therapeutic use. This first-in-human study of EB-001, a novel purified form of Botulinum toxin type E administered IM, fulfilled its objectives of evaluating the safety, tolerability, and efficacious dose-range of EB-001. A dose response was observed, with greater proportions of treatment responders in the higher dosing cohorts of EB-001. An IR-2 response was observed starting with Cohort 3 and increased in higher dose cohorts, suggesting that the efficacious dose range of EB-001 may be at doses used in Cohorts 4 to 7. Cohorts 6 and 7 had 80% IR-2 responders, a response rate similar to approved Botulinum toxin type A products. Subjects achieving none or mild FWS grades were observed starting at Cohort 2. In terms of onset of effect, treatment response was observed as early as 24 hours following dosing, which supports prior reports suggesting that Botulinum toxin type E has a faster onset than type A.

Regarding the duration of effect defined as the proportion of responders with a none or mild rating, an effect was observed through Day 14 in 1 subject in most of the 5 higher dose cohorts, and through Day 30 in 1 subject in 2 of the 5 higher dose cohorts. All doses of EB-001 showed good tolerability with no local injection site reactions. There were no SAEs or severe TEAEs reported, and no discontinuations due to a TEAE. The most common TEAE of headache was mild or moderate in severity, and there were no other treatment related AEs. There were no events of ptosis at any dose levels, and no events potentially related to spread of toxin. Therefore, the clinical safety and tolerability profile seems favorable in this study. The efficacy and safety profiles of EB-001 are promising and support the potential of EB-001 as a unique treatment option in the treatment of GL and other facial aesthetic uses. The fast onset can fulfill an unmet need for individuals seeking a rapid treatment for facial wrinkles before unexpected social or professional events. The limited duration of effect can be beneficial for individuals who may be considering first time use of a Botulinum neurotoxin treatment, and are unwilling to make a longer-term commitment. An EB-001 treatment would allow them to assess the aesthetic effect over a shorter duration of effect compared with the 12-week duration of effect of Botulinum toxin type A products. In this first clinical study in subjects with GL, EB-001 showed favorable safety and tolerability in all cohorts. Five out of the 7 cohorts showed numerically higher response rates compared to placebo, supporting the efficacy of EB-001 in the reduction of GL severity. The 2 highest doses provided an 80% response rate, similar to approved Botulinum toxin type A products. In contrast to the known time course of type A products, the clinical effect of EB-001 was seen within 24 hours (onset) and lasted between 14-30 days (duration). This differentiated clinical profile supports the future development of EB-001 for facial aesthetic and key therapeutic uses, where fast onset and short duration of effect are desirable.

TABLE S-1
Dose Escalation Scheme

| | Total EB-001 | Dose at | Doses at Medial | Dose at Lateral |
|---|---|---|---|---|
| Cohort¹ | Dose (ng)² | Procerus (ng) | Corrugators (ng) | Corrugators (ng) |
| 1 | 0.1 | EB-001 | EB-001 into right and left | EB-001 into right and left |
| | | (0.02) | corrugators (0.02 each) | corrugators (0.02 each) |
| 2 | 0.3 | EB-001 | EB-001 into right and left | EB-001 into right and left |
| | | (0.06) | corrugators (0.06 each) | corrugators (0.06 each) |
| 3 | 0.9 | EB-001 | EB-001 into right and left | EB-001 into right and left |
| | | (0.18) | corrugators (0.18 each) | corrugators (0.18 each) |
| 4 | 1.2 | EB-001 | EB-001 Into right and left | EB-001 into right and left |
| | | (0.24) | corrugators (0.24 each) | corrugators (0.24 each) |
| 5 | 1.6 | EB-001 | EB-001 into right and left | EB-001 into right and left |
| | | (0.32) | corrugators (0.32 each) | corrugators (0.32 each) |
| 6 | 2.1 | EB-001 | EB-001 into right and left | EB-001 into right and left |
| | | (0.42) | corrugators (0.42 each) | corrugators (0.42 each) |
| 7 | 2.8 | EB-001 | EB-001 into right and left | EB-001 into right and left |
| | | (0.56) | corrugators (0.56 each) | corrugators (0.56 each) |

### Example 7—Use of Botulinum Toxin Type E to Treat Crows Feet

A 57 year old neurotoxin-naive man with crow's feet resulting from years of sun exposure seeks treatment from his physician. The physician recommends a composition as disclosed herein, which is injected sub-dermally on either side of the patient's eyes. Each injection site receives about 3 units of type E botulinum toxin, with several injections made on either side of the eye. The crow's feet disappear within about 2 days after treatment, and remain reduced for two months.

### Example 8—Use of Botulinum Toxin Type E for Brow Lift

A 60 year old neurotoxin-naive woman presents with eyebrows extending below her brow bone. The physician recommends a composition as disclosed herein, which is injected subdermally above each eye. Each injection site receives about 10 units of type E botulinum toxin, with several injections made on either side of the eye. The drooping of the brow is reduced within about 2 days, and is substantially alleviated for 3 months after administration.

### Example 9—Use of Botulinum Toxin Type E for Breast Augmentation

A 30 year old neurotoxin-naive woman elects breast augmentation surgery. 4 hours prior to the procedure, botulinum toxin type E is administered in the proximity of where the surgical incisions will be made. The administration reduces muscle tension in the area of the incision, resulting in minimal scarring. Two weeks after the procedure, a supplemental dose is administered.

### Example 10—Use of Botulinum Toxin Type E for Breast Reconstruction

A 30 year old neurotoxin-naive woman elects breast reconstruction surgery. 14 hours prior to the procedure, botulinum toxin type E is administered in the proximity of where the surgical incisions will be made. The administration reduces muscle tension in the area of the incision, resulting in minimal scarring. Two weeks after the procedure, a supplemental dose is administered.

### Example 11—Use of Botulinum Toxin Type E to Treat Episiotomy

A 44 year old neurotoxin-naive woman undergoes an episiotomy. Immediately after the procedure, 4 ng of type E botulinum toxin to the tissue surrounding surgical area. Within 20 hours, muscle and nerve activity surrounding the wound is greatly reduced.

### Example 12—Use of Botulinum Toxin Type E to Treat a Sports Hernia

A 28 year old neurotoxin-naive man suffers a sports hernia. His doctor administers 4 ng of type E botulinum toxin to the tissue surrounding both the hernia. Within 10 hours, muscle and nerve activity surrounding the injury is greatly reduced.

### Example 13—Use of Botulinum Toxin Type E to Treat a Shoulder Separation

A 48 year old neurotoxin-naive man suffers a shoulder separation. His doctor administers 4 ng of type E botulinum toxin to the tissue surrounding both the hernia. Within 16 hours, muscle and nerve activity surrounding the injury is greatly reduced.

### Example 14—Use of Botulinum Toxin Type E to Treat a Torn ACL

A 23 year old woman suffers a torn ACL. 6 hours after the injury, her doctor administers 7 ng of type E botulinum toxin to the muscle surrounding the torn ligament. Within 30 hours, muscle and nerve activity surrounding the wound is greatly reduced.

### Example 15—Use of Botulinum Toxin Type E to Treat Axillary Hyperhidrosis

A 44-year old neurotoxin-naive male complains of excessive axillary perspiration. His doctor diagnoses axillary hyperhidrosis, and prescribes injections of botulinum type E to provide rapid relief. The injections are made subcutaneously (s/c) to the underarm in a grid-like pattern approximately every 1-2 cm apart. Each injection contains 10 units of type E neurotoxin. The injection provides a rapid effect (with 48 hours) of a limited duration (two weeks).

After the two-week evaluation period, the patient decides whether to undergo a supplemental injection of a fast-acting neurotoxin or further treatment with a longer-acting neurotoxin.

### Example 16—Use of Botulinum Toxin Type E to Treat Neuropathic Pain

A 42 year old neurotoxin-naive man experiences neuropathic diabetes pain in his feet. To alleviate the patient's symptoms, botulinum type E is injected into the vicinity of nerves in the feet. The patient experiences reduced pain.

### Example 17—Use of Botulinum Toxin Type E in Connection with Treatment of a Broken Led

A 57 year old man suffers a compound leg fracture in an automobile accident. First responders stabilize the patient. 24 hours prior to corrective surgery, the patient's doctor administers 10 ng of type E botulinum toxin to the muscles and nerves on both sides of the fracture. Within 24 hours, pain sensation in the area surrounding the fracture is greatly reduced. At the time of surgery, the doctor also administers a local anesthetic to the area.

### Example 18—Use of Botulinum Toxin Type E in Connection with Treatment of a Broken Arm

An 18 year old man suffers an arm fracture in a football game. First responders stabilize the patient. 16 hours prior to corrective surgery, the patient's doctor administers 6 ng of type E botulinum toxin to the muscles and nerves on both sides of the fracture. The patient is also administered an opioid. Within 16 hours of the neurotoxin administration, pain sensation in the area surrounding the fracture is greatly reduced.

### Example 19—Use of Botulinum Toxin Type E in Connection with Dental Surgery

A 22 year old woman is scheduled for a root canal. 20 hours prior to corrective surgery, the patient's dentist administers 4 ng of type E botulinum toxin to the nerves on both sides of the affected area. Within 20 hours of the neurotoxin administration, pain sensation in the area surrounding the area is greatly reduced.

### Example 21—Use of Botulinum Toxin Type E in Connection with Rhinoplasty

A 38 year old man is scheduled for a rhinoplasty procedure. 24 hours prior to corrective surgery, the patient's doctor administers 6 ng of type E botulinum toxin to the muscles and nerves on both sides of the treatment area. Within 24 hours of the neurotoxin administration, pain sensation in the area surrounding the treatment area is greatly reduced. At the time of surgery, a local anesthetic is administered around the patient's nose. After the surgery, the doctor also administers an opioid to the patient.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A method for reducing pain, comprising administering a therapeutically effective amount of an opioid and a therapeutically effective amount of a fast-acting botulinum neurotoxin.

2. The method of claim 1, wherein the administered dose comprises an amount between 2 and 20 nanograms of neurotoxin followed by administration of between 1 and 100 mg of the opioid.

3. The method of claim 2, wherein said administration comprises injection of the neurotoxin and oral administration of the opioid.

4. The method of claim 2, wherein said method further comprises administration of an intermediate-acting neurotoxin to the patient.

5. The method of claim 2, wherein said method further comprises administration of a long-acting neurotoxin to the patient.

6. The method of claim 1, wherein said pain comprises post-surgical pain.

7. A method for reducing pain, comprising administering a therapeutically effective amount of a bupivacaine and a fast-acting botulinum neurotoxin.

8. The method of claim 7, wherein the administered dose comprises an amount between 2 and 20 nanograms of neurotoxin followed by injection of between 1 and 175 mg bupivacaine.

9. The method of claim 8, wherein said administration comprises two separate injections.

10. The method of claim 8, wherein said method further comprises administration of an intermediate-acting neurotoxin to the patient.

11. A method for administering to the patient a therapeutically effective amount of;
a fast-acting neurotoxin; and
bupivacaine;
to a patient in an area where treatment is desired.

12. A method for initiating a neurotoxin treatment in a neurotoxin-naive patient, comprising administering to the patient a therapeutically effective amount of a fast-acting neurotoxin to a patient in an area where treatment is desired.

13. The method of claim 12, wherein said fast-acting neurotoxin comprises botulinum neurotoxin serotype E.

14. The method of claim 13, wherein said therapeutically effective amount comprises an amount of between about 0.2 and 60 ng.

15. The method of claim 14, wherein said therapeutically effective amount comprises an amount of between about 0.2 and 40 ng.

16. The method of claim 15, wherein said therapeutically effective amount comprises an amount of between about 0.2 and 20 ng.

17. A composition comprising at a combination of botulinum neurotoxin serotype A (BoNT/A) and botulinum neurotoxin serotype E (BoNT/E).

18. The composition of claim 17, wherein the BoNT/E is at least half the activity of BoNT/A.

19. The composition of claim 17, wherein the BoNT/A and BoNT/E are lyophilized separately and then placed together in a single vial for reconstitution.

20. A method for reducing muscle activity, comprising administering a composition according to claim 19.
